(19) United States
(12) Patent Application Publication
(10) Pub. No.: US 2006/0110405 A1
(43) Pub. Date: May 25, 2006

(54) Plasma or serum fraction for treatment and prevention of viral infections and related conditions

(76) Inventor: Robert W. Buckheit JR., Myersville, MD (US)

Correspondence Address: KING & SPALDING LLP, 1180 PEACHTREE STREET, ATLANTA, GA, 30309, US

(21) Appl. No.: 11/209,571

(22) Filed: Aug. 22, 2005

Related U.S. Application Data
(60) Provisional application No. 60/603,383, filed on Aug. 20, 2004.
(60) Provisional application No. 60/672,732, filed on Apr. 19, 2005.
(60) Provisional application No. 60/673,534, filed on Apr. 21, 2005.
(60) Provisional application No. 60/673,533, filed on Apr. 21, 2005.
(60) Provisional application No. 60/673,714, filed on Apr. 21, 2005.
(60) Provisional application No. 60/673,713, filed on Apr. 21, 2005.

(51) Int. Cl. C12Q 1/68; A61K 39/12

(52) U.S. Cl. 424/204.1; 435/6

(57) ABSTRACT

The present invention describes compositions and methods for the treatment and/or prevention of viral infections and related conditions, including HIV infections.

### CROSS REFERENCE TO RELATED APPLICATION

This application claims priority to U.S. Provisional Patent Application No. 60/603,383 filed on Aug. 20, 2004, U.S. Provisional Patent Application No. 60/672,732 filed on Apr. 19, 2005, U.S. Provisional Patent Application No. 60/673,534 filed on Apr. 21, 2005, U.S. Provisional Patent Application No. 60/673,533 filed on Apr. 21, 2005, U.S. Provisional Patent Application No. 60/673,714 filed on Apr. 21, 2005, and U.S. Provisional Patent Application No. 60/673,713 filed on Apr. 21, 2005, the contents of all of which are incorporated herein by reference.

### FIELD OF THE INVENTION

The present invention provides compositions, methods and uses for treating and preventing viral infections and related conditions, and in one embodiment for treating and preventing infection with the human immunodeficiency virus (HIV).

### BACKGROUND OF THE INVENTION

Infectious disease generally, and viral infection in particular, is a leading cause of death worldwide. The burden of viral infection includes both illness and death among the infected, as well as the enormous financial burdens placed on society. The threat to economic and political stability posed by viral infection is particularly great in the developing world, which has been disproportionately effected by viral infection. The threat of emerging and re-emerging viral diseases remains particularly great.

Among the greatest viral threats to public health is HIV. Since the first case reports of the AIDS in the early 1980's in the United States, more than 60 million people worldwide have been infected with HIV, and currently more than 40 million people are estimated to be living with HIV/AIDS. Deaths among those already infected will continue to increase for some years even if prevention programs reduce the number of new infections. AIDS is increasingly a disease of the young and the poor, producing entire generations of orphans in developing nations.

Identification of HIV as the etiologic agent of AIDS led to the development of drugs for its treatment. As early as 1986, preliminary reports on the use of 3′-azido-3′-deoxy-thymidine were presented at the Second International AIDS Conference. That drug (AZT, Zidovudine, Retrovir) became the first therapeutic approved by the Food and Drug Administration (FDA) for use in treatment of HIV. Other therapeutics quickly followed including reverse transcriptase inhibitors (i.e., nucleoside reverse transcriptase inhibitors or NRTIs, non-nucleoside reverse transcriptase inhibitors or NNRTIS), protease inhibitors, fusion inhibitors, and antisense drugs.

Hepatitis C virus (HCV) is another threat to public health. First identified in 1988, HCV is now known to have infected more than 170 million persons worldwide, including more than 4 million people in the United States. Chronic hepatitis C infection (CHCV) can persist for years, leading to chronic liver disease, liver failure or liver cancer. HCV is now responsible for the majority of liver transplants in the United States (Davis G L. Reviews in Gastroenterological Disorders (2004) 4(1): 7-17) Direct and indirect healthcare costs associated with HCV disease in the United States alone have been estimated to be in the billions of dollars annually (Leigh J P et al. Arch Intern Med. (2001) 8:161(18):2231-7). There is no vaccine to prevent spread of the disease, and currently available therapies are expensive, often poorly tolerated, and ineffective in a substantial percentage of infected patients.

Hepatitis B virus (HBV) infection has also reached epidemic levels worldwide, with one in every three people has been infected with the virus globally. Chronic HBV infection (CHB) can lead to severe liver disease including cirrhosis, liver failure, liver cancer and death, and more than 400 million people are chronically infected worldwide. The World Health Organization (WHO) estimates that HBV infections cause more than one million deaths every year. Availability of an HBV vaccine has failed to prevent global spread of the disease.

Respiratory viruses also threaten public health. While common in all age groups, respiratory infections are typically limited to the upper respiratory tract (URT). The infections can spread to the lower respiratory tract (LRT), where they can result in more serious disease and death. The most clinically significant respiratory viruses include the influenza viruses, respiratory syncytial viruses (RSV), parainfluenza viruses, rhinoviruses, adenoviruses and coronaviruses. Influenza is the most severe of the more common respiratory viral infections, responsible for the deaths of more than 36,000 people and 114,000 hospitalizations in the United States each year. Moreover, the threat of an influenza pandemic is ever-present, with the cost of such an event in the United States alone estimated to range from $71.3 billion to $166.5 billion dollars (Meltzer M et al. Emerging Infectious Diseases (1999) vol. 5). Only limited progress has been made in developing anti-virals for the treatment of respiratory infections. As a result, most current treatment is limited to supportive therapy (e.g., analgesics and antihistamines) and prevention. Vaccines are available for influenza viruses and are being developed for others, but effectiveness can be limited by high rates of mutation and antigenic shift.

Herpes viruses are also a leading cause of human viral disease, second only to influenza and cold viruses. The herpes simplex virus (HSV) affects more than one third of the world's population and is responsible for a wide array of human disease. Overall, mortality is most closely associated with perinatal infection, encephalitis, and infection in the immunoincompetent host. Since there is no cure for HSV, the goals of pharmacotherapy are to reduce morbidity and to prevent complications. Generally, medical treatment of HSV currently revolves around specific antiviral treatment, such as nucleoside analogs. Because of the possibility of developing drug-resistant viral mutations, such as thymidine kinase-negative mutants, the use of antiviral therapeutics must be tempered against the potential for resistance.

Immunologically-Based Therapeutic Approaches to Viral Infection

While efforts continue to develop new anti-viral drugs, or combinations of such drugs, there is a need for different therapeutic approaches to viral infections. Attention has turned to defensive, immune-based strategies. Passive immunotherapy, the use of neutralizing antibodies produced by another organism, has received some attention as a possible therapeutic for certain viral infections such as HIV. It differs from active immunotherapy or vaccination, which typically involves exposing the patient to an antigen so that the patient will form his or her own antibodies in response. Historically, passive immunotherapy has been used to treat a variety of diseases.

U.S. Pat. No. 4,863,730 (Karpas) teaches a method for treating HIV by administering heterologous human neutralizing antibodies. Plasma is obtained from HIV positive patients and then processed to provide a preparation having a high titer of neutralizing antibodies. While some clinical reports suggest therapeutic benefit (i.e., reduced HIV viremia and delay in onset of disease), this method is not considered broadly applicable and large scale production may be a problem.

WO 97/02839 (Davis) teaches methods and compositions for treating viral infections involving administration of neutralizing antibodies produced in goats. The goats are immunized with viral lysates, such as HIV or influenza. The blood of the immunized animal is then collected, and processed by standard extraction and purification methods (e.g., ammonium sulfate precipitation followed by dialysis or gel filtration) to produce a composition enriched for heterologous neutralizing antibodies. Other filings by Davis include WO 01/60156, U.S. patent application Publication No. 20020086022 and WO 02/07760. All teach the processing of immunized animal sera to obtain immunoglobulin-enriched serum extracts suitable for in vivo use. The Davis method and composition have been used to treat HIV patients outside of the United States, as widely reported in such media sources as the Washington Post (Apr. 9, 2000) and on Dateline Houston (Sep. 18, 1998).

U.S. Pat. No. 6,670,181 (Gelder et al.) discloses methods and compositions for the treatment of viral infections, specifically HIV infections, directed to neutralizing antibodies produced in goats. These neutralizing antibodies are said to recognize certain viral epitopes that fail to elicit neutralizing antibodies in humans when encountered through natural infection. The neutralizing antibody composition of Gelder et al. corresponds to HRG214, a ployclonal antibody preparation produced by immunization of goats with purified HIV proteins from HIV-1 MN and HIV-2 NZ, followed by booster immunizations with synthetic peptides from highly conserved HIV epitode regions, mapping to HIV-1 SF2, env, and gag. HRG214 is manufactured by Vironyx Corporation and was recently the subject of a Phase I clinical trial (Pett SL et al., HIV Clin Trials. (2004) 5(2):91-8). See also U.S. Pat. No. 6,335,017, U.S. Pat. No. 6,258,599, U.S. Pat. No. 6,043,347, as well as U.S. patent application Publication Nos. 20020086034 and 20040141996.

A number of monoclonal antibodies have been identified that broadly neutralize HIV (see, e.g., Burton D R et al. Science (1994) 266: 1024-1027; Trkola A. et al. J. Virol. (1996) 70: 1100-1108; Conley A J. et al. Proc. Natl. Acad. Sci. USA (1994) 91: 3348-3352; Sullivan N et al. J. Virol. (1998) 72: 4694-4703; Moulard M et al. Proc. Natl. Acad. Sci. USA (2002) 99: 6913-6918).

Overall, however, the use of neutralizing antibodies and immunoglobulin concentrates for the treatment of HIV has proved disappointing. Early clinical results found no clear benefits for the majority of patients treated (Stiehm, E R et al. Summary of the workshop on passive immunotherapy in the prevention and treatment of HIV infection. The Passive Antibody Workshop Participants. Clinical Immunology and Immunopathology (1995) 75L84-93). Most antibodies produced against HIV are unable to neutralize primary isolates. Although monoclonal antibodies demonstrate a high antiviral potential in vitro, some doubt their usefulness for in vivo applications (Steigler G. Journal of Antimicrobial Chemotherapy (2003) 51: 757-759). In general, heterologous neutralizing antibodies are thought to be more useful in preventing HIV than affecting the course of disease progression in patients already infected with the virus (Montefiori D. et al. J Virol (2000) 75: 10200-10207).

WO 03/004049 (Dalgleish) speculates that the therapeutic activity of goat serum processed in the manner of Davis et al. may actually be dependent on anti-HLA and/or anti-FAS antibodies. Dalgleish suggests that the anti-inflammatory effect of these antibodies prevents over-stimulation of the immune system by viral epitopes (gp 120), which resemble normal human HLA. Dalgleish speculates that other antibodies (e.g., antibodies to the dopamine receptor) may play a role in modulating the beneficial therapeutic effects of the immunoglobulin extract, alone or in combination with anti-HLA and anti-FAS. These compositions are said to be useful in treating certain diseases with inappropriately high HLA levels, including HIV, multiple sclerosis, diabetes, and certain forms of cancer. See also WO 03/064472 (Dalgleish).

WO 04/033665 (Tolett), published Apr. 22, 2004, discloses a heterologous plasma cocktail for treatment of HIV using the filtered, but otherwise unpurified, serum or plasma of HIV-exposed animals. The plasma or serum cocktail is derived by animal blood which has not been subject to any technique designed to purify, separate, isolate or concentrate any component of the plasma or serum.

Given the serious heath effects caused by viral infections, there exists a need for new therapeutic strategies.

Therefore, it is an object of the present invention to provide a composition for the treatment or prevention of viral infections and related disorders.

It is another object of the present invention to provide a method and use for treating and preventing viral infections and related disorders that consists of a simple regimen of therapy, encourages compliance with the therapy and has minimal side effects.

It is a further object of the present invention to provide a use in the manufacture of a composition or medicant for the treatment and prevention of viral infections and related disorders.

### SUMMARY OF THE INVENTION

A composition, method and use for the treatment or prevention of viral infections and related disorders in a subject, such as a human, is disclosed. The composition, method and use provide a plasma or serum fraction depleted of high molecular weight proteins or biological agents, which fraction is derived from a mammal exposed to an inoculate, such as an HIV-bearing inoculant. Without being bound by any particular theory, it is believed that one or more biological agents present in the plasma or serum fraction depleted of high molecular weight proteins or biological agents generates a beneficial plethoric effect anti-viral in vivo. It is also believed that these biological agent(s) have anti-viral efficacy even when generated with the use of an unrelated viral inoculant. In other words, the modified plasma or serum can be generated, for example, by exposure to HIV, and then used therapeutically to treat a patient infected with a different virus.

Accordingly, one aspect of the present invention is a composition which is a plasma or serum fraction derived from a mammal exposed to an inoculant, which fraction has been depleted of two or more high molecular weight proteins or biological agents.

The high molecular weight protein(s) or biological agent(s) depleted from the plasma or serum fraction of the present invention may vary. Representative, non-limiting high molecular weight proteins include immunoglobulin (e.g., IgG, IgM), albumin, transferrin, haptoglobin and lipoproteins. In a particular embodiment of the present invention, the composition is a plasma or serum fraction derived from a mammal exposed to an inoculant, which fraction has been depleted of immunoglobulin and albumin.

The term “depleted” is used to indicate a reduction in the amount of a high molecular weight protein(s) or biological agent(s) in a given sample after the sample is treated according to the method of the present invention. In one embodiment of the present invention, the sample is depleted of 100%, 99%, 98%, 97%, 96%, 95%, 94%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 56%, 54%, 53%, 52%, 51%, 50%, 49-45%, 44-40%, 39-35%, 34-30%, 29-20%, 19-10%, 10%-5%, 5%-1% of the high molecular weight protein(s) or biological agent(s) present in the original sample.

The inoculant used to generate the plasma or serum fraction of the present invention may vary. In one embodiment, the inoculant is a viral inoculant. Viral inoculants include, without limitation, HIV-bearing inoculants, HBV-bearing inoculants, HCV-bearing inoculants, respiratory virus-bearing inoculants and herpes virus-bearing inoculants. In a further embodiment, the inoculant is a non-viral inoculant such as a bacterial inoculant.

In a particular embodiment, the present invention is composition useful for the treatment or prevention of HIV infections or related conditions which is a serum fraction derived from a mammal exposed to an HIV-bearing inoculant, which fraction has been depleted of immunoglobulin and albumin.

In a particular embodiment, the mammal used to generate the plasma or serum fraction of the present invention is a goat.

Another aspect of the present invention is a composition useful in the treatment of a viral infection or related conditions which is a plasma or serum fraction derived from a mammal exposed to an inoculant, which fraction has been depleted of one or more high molecular weight proteins or biological agents, wherein the viral infection is selected from the group consisting of HBV viral infections, HCV viral infections, respiratory viral infections and herpes viral infections.

A further aspect of the present invention is a composition which is a plasma or serum fraction derived from a mammal exposed to an inoculant, which fraction has been depleted of proteins or biological agents with a molecular weight greater than approximately 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 kD.

In a particular embodiment, the composition is a plasma or serum fraction derived from a mammal exposed to an inoculant, which fraction has been depleted of proteins with a molecular weight greater than approximately 30 kD.

In a further particular embodiment, the composition is a plasma or serum fraction derived from a mammal exposed to an inoculant, which fraction which has been depleted of proteins with a molecular weight greater than approximately 50 kD.

Another aspect of the present invention is a method of preparing the composition of the present invention involving (a) exposing a mammal to an inoculant; (b) allowing time for the mammal to respond to the inoculant and to produce one or more beneficial biologic agents in the blood; (c) obtaining the plasma or serum; and (d) processing the plasma or serum to isolate the anti-viral activity from two or more high molecular weight proteins or biological agents present in the unprocessed plasma or serum.

In a particular embodiment, the mammal is not susceptible to infection with the inoculant.

The process used to isolate the anti-viral activity (e.g., HIV) from the one or more high molecular weight proteins or biological agents present in the unprocessed plasma or serum may vary. The process may include, without limitation, fractionation methods such as fractional precipitation, dialysis and ultrafiltration, and/or chromatographic fractionation. The process may involve a single fractionation step or multiple fractionation steps, involving the same or different fractionation methods.

Another aspect of the present invention is a method of treating or preventing viral infections or related conditions in a subject such as a human by administering a therapeutic amount of a plasma or serum-fraction derived from a mammal exposed to an inoculant, which fraction has depleted of two or more high molecular weight proteins or biological agents, either alone or in combination or alternation with another anti-viral agent or agent that treats a related condition.

In a particular embodiment, the subject in need thereof is a human.

The composition of the present invention can be administered by any effective means, including but not limited to, subcutaneous, parenteral, intravenous, intraarterial or oral administration. In a particular embodiment, the composition is administered by subcutaneous injection.

In one embodiment, the present invention is a method of treating or preventing an HIV infection or related conditions in a subject such as a human by administering a therapeutic amount of a serum-fraction derived from a mammal exposed to an HIV-bearing inoculant, which fraction has been depleted of immunoglobulin and albumin, either alone or in combination or alternation with another anti-HIV agent or agent that treats a related condition (e.g., an opportunistic infection).

In another particular embodiment, the present invention is a method of treating or preventing a respiratory virus infection or related conditions in a subject such as a human by administering a therapeutic amount of a serum-fraction derived from a mammal exposed to an viral-bearing inoculant, which fraction has depleted of immunoglobulin and albumin, either alone or in combination or alternation with another anti-respiratory virus agent or agent that treats a related condition (e.g., an opportunistic infection). In a particular embodiment, the respiratory virus infection is an influenza a, influenza b, RSV or SARS infection.

In another embodiment, the present invention is a method of treating or preventing a viral infection or related condition by administering a therapeutic amount of a plasma or serum-fraction derived from a mammal exposed to an inoculant, which fraction has been depleted of one or more different high molecular weight proteins or biological agents, either alone or in combination or alternation with another anti-viral agent or agent that treats a related condition (e.g., an opportunistic infection), wherein the viral infection is selected from the group consisting of HBV infections, HCV infections, respiratory viral infections or herpes viral infections.

### DETAILED DESCRIPTION OF THE DRAWINGS

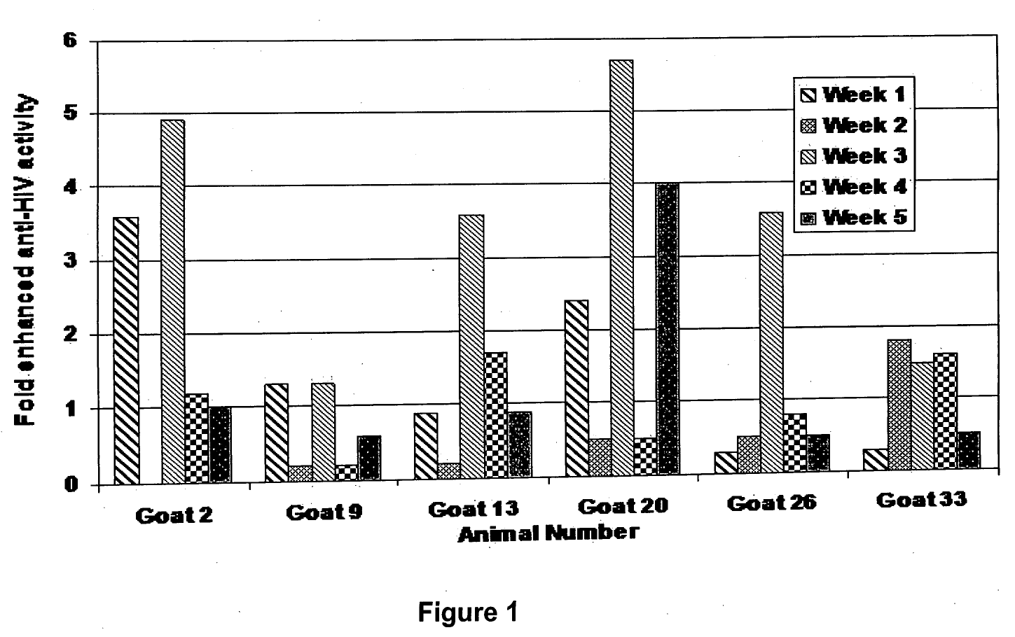

FIG. 1 is a graphical representation of the ratio of anti-HIV activity of weekly samples from goats inoculated with plasma from an HIV-infected donor, as described in Example 10. Animals were inoculated with 5 ml of plasma from an HIV-infected donor. Serum was prepared from blood drawn prior to inoculation and at weekly intervals for 5 weeks following inoculation. Anti-viral activity was assessed using the HIV-β-galactosidase assay and the results were expressed as the fold-increase in activity (decrease in EC50) of the weekly serum samples relative to the pre-inoculation serum (week 0). Week 2 sample for Goat 2 was not analyzed.

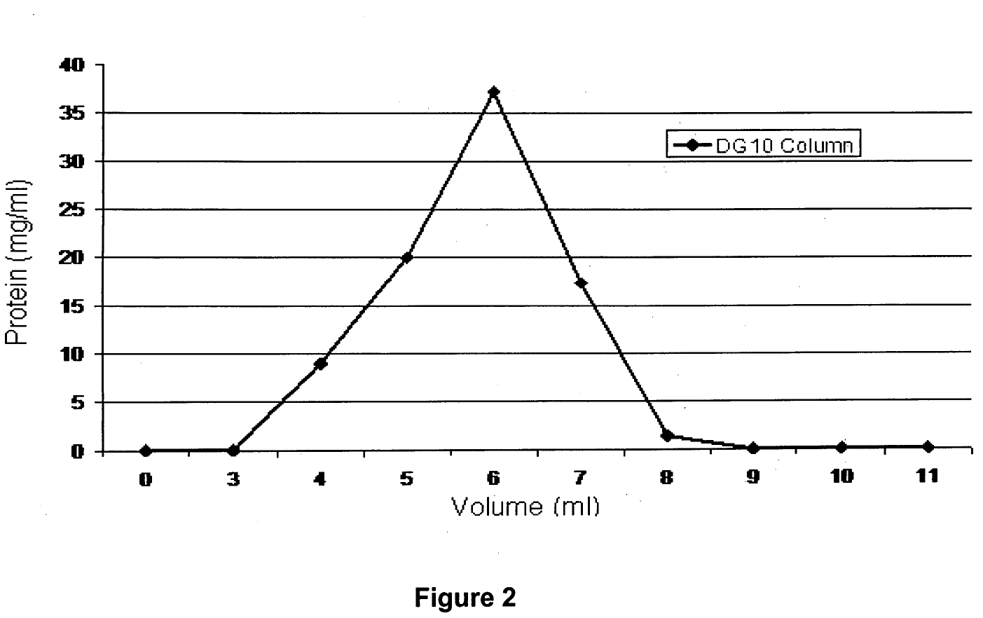

FIG. 2 is a graphical representation of the protein profile obtained as described in Example 11 from a DG-10 desalting column of Week 3 serum from an animal inoculated as described in Example 10. The column was equilibrated with buffer A, 3 ml of serum was loaded onto the column and 1 ml fractions were collected. The protein concentration of each fraction was determined and the results were plotted vs. the approximate elution volume. Fractions representing milliliters 4 through 9 were pooled for DEA-blue chromatography.

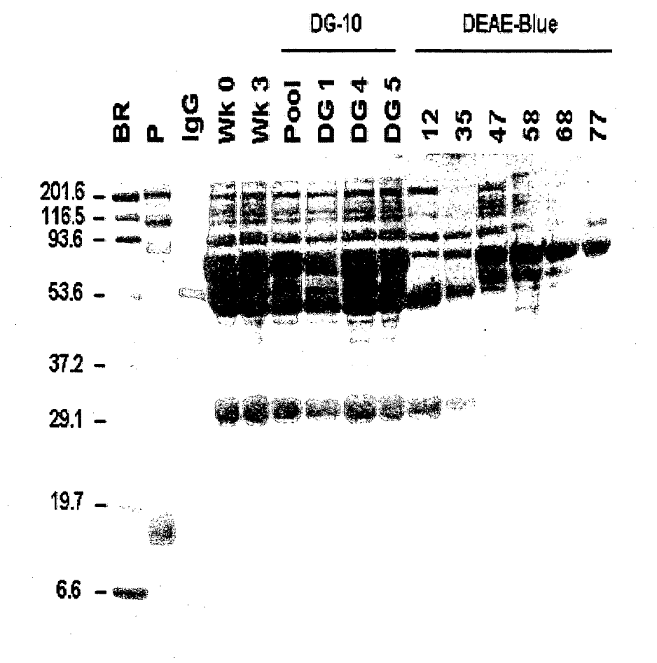

FIG. 3 shows Coomassie blue stain of partially purified protein serum fractions. Protein from serum and partially purified fractions (as described in Example 11) was subjected to electrophoresis on a 6 to 18% polyacrylamide Tris-SDS gel. Following electrophoresis, the gel was stained with Coomassie G-250 to visualize the proteins. BR and P are Bio Rad broad range pre-stained molecular weight markers, and Pierce TriChromRanger molecular weight markers, respectively. The molecular weight in kilodaltons of the BioRad markers are indicated on the left hand side of the figure. IgG is 2 μg of purified goat IgG obtained from the NIH AIDS Research and Reference Reagent Program.

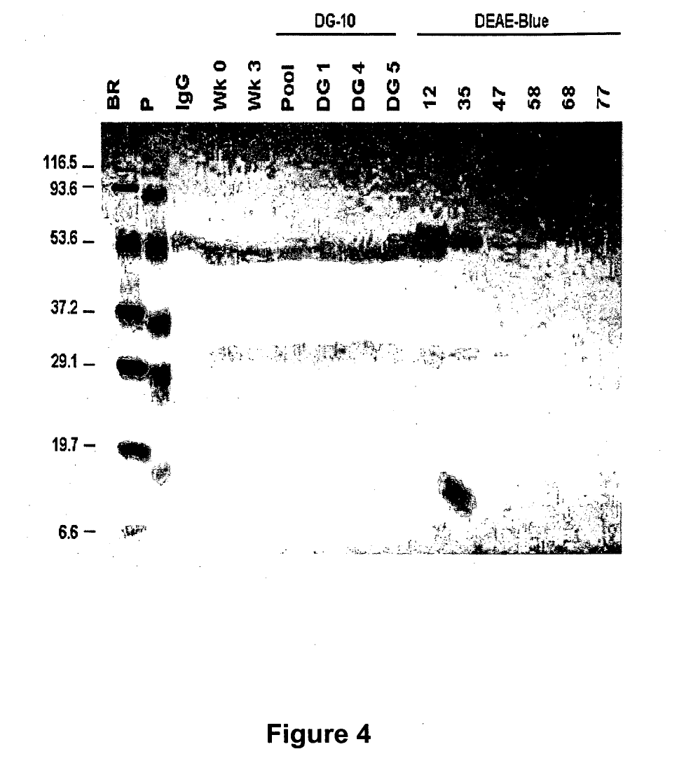

FIG. 4 is an immunoblot of partially purified serum fractions with anti-goat IgG, as described in Example 11. Protein from serum and partially purified fractions was subjected to electrophoresis on a 6 to 18% polyacrylamide Tris-SDS gel. Following electrophoresis, the gel was stained with coomassie G-250 to visualize the proteins. BR and P are Bio Rad broad range pre-stained molecular weight markers, and Pierce TriChromRanger molecular weight markers, respectively. The molecular weight in kilodaltons of the BioRad markers are indicated on the left hand side of the figure. IgG is 2 μg of purified goat IgG obtained from the NIH AIDS Research and Reference Reagent Program.

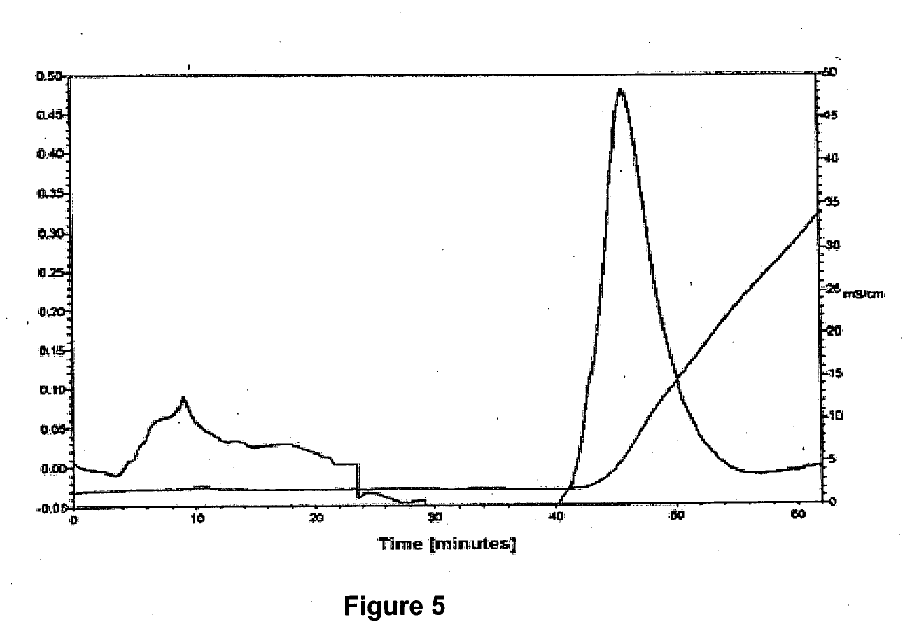

FIG. 5 is a graphical representation of the chromatographic profile for the DEAE-Blue column fractionation of the 66% ammonium sulfate pellet detailed in Example 12. The top trace monitors absorbance at 254 nm vs time and represents the protein elution profile. The bottom trace monitors eluate conductivity vs. time and represents the ionic concentration of the wash or elution buffer.

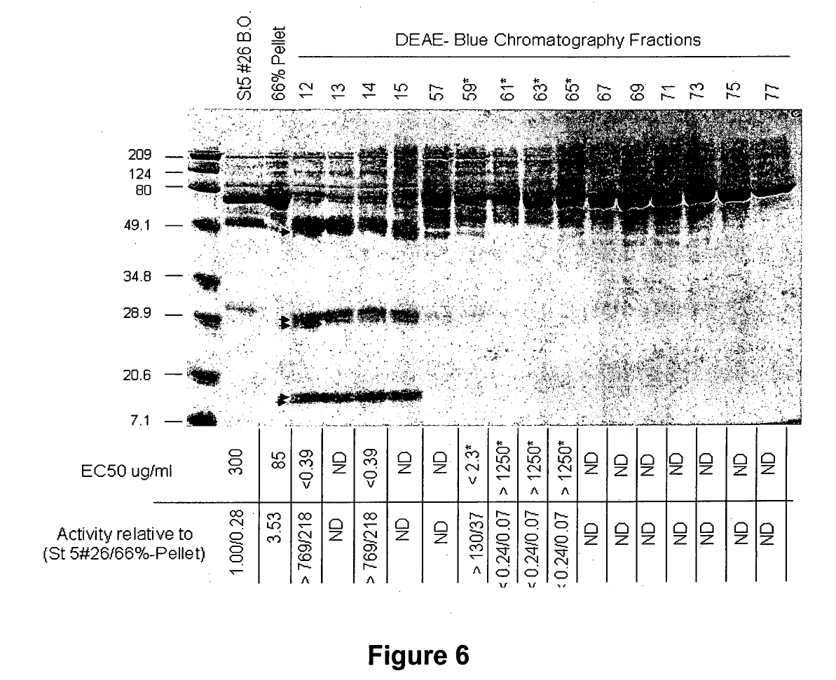

FIG. 6 shows SDS-PAGE and inhibition of HIV attachment from the partial fractionation detailed in Example 12. Five micrograms of total protein for each fraction was electrophoresed on a 8-16% polyacrylamide gel. The gel was stained with Bio Safe Commassie stain over night at room temperature and destained with water. The image was captured using an Alpha Inotech gel imager. The molecular weights of dye-labeled protein standards (BioRad) is indicated on the left of the figure. The relative activity of each analyzed fraction was calculated by dividing the EC50 of the starting material (66% Pellet) by the EC50 of the fraction. The arrows indicate five proteins that are enriched in the active flow through fractions relative to the 66% pellet.

### DETAILED DESCRIPTION OF THE INVENTION

The present invention includes compositions, methods and uses for treating and preventing viral infections and related conditions. In a particular embodiment, the compositions and methods of the present invention are useful for treating or preventing HIV infection, influenza A infection, influenza B infection, RSV infection, SARS infection and related conditions. In addition, these compounds or formulations can be used prophylactically to prevent or retard the progression of clinical illness in individuals who are positive for viral antibodies or antigens, or who have been exposed to a virus.

The composition and methods of the present invention can be used to treat or prevent a viral infection which is responsive to such treatment or prevention. Representative, non-limiting examples of viral infections that can be treated or prevented according to the present invention include those infections cause by viral agents listed in Section A (“Process for Producing the Plasma or Serum Fraction”) below as suitable viral inoculants. In a particular embodiment, the compositions and methods of the present invention can be used to treat or prevent HIV infections, HBV infections, HCV infections, respiratory virus infections and herpes virus infections.

A. Process for Producing the Plasma or Serum Fraction

(i) Selection of Animal

In one embodiment, the plasma or serum fraction of the present invention may be prepared using any mammal not susceptible to infection with the inoculant (e.g., not susceptible to infection with HIV) that produces an effective product on inoculation according the process described in detail herein. Non-limiting examples of mammals for use in producing the plasma or serum fraction of the present invention include rabbit, ovine, bovine, canine, feline, equine, murine and the like, goats (for example Bora goats), lamb, sheep, horses, deer, pigs, cows, sheep, chickens, dogs, cats, and rabbits. In a particular embodiment, the mammal is an ungulate or hoofed-mammal. Non-limiting examples of ungulates include goats, sheep, horses, and cows. In one particular embodiment, the mammal is a goat.

(ii) Inoculation of the Animals

Any inoculate suitable for generating the plasma or serum fraction is suitable for use in the present invention. The inoculant may contain a single immunogen or multiple immunogens. The inoculant may be, for example, a viral inoculant or a non-viral inoculant (e.g., a bacterial inoculant).

In one embodiment of the present invention, the viral inoculant is an HIV-bearing inoculant. In another embodiment of the present invention, the viral inoculant is a Herpesviridae bearing inoculant, a Retroviridae bearing inoculant, a Flaviviridae bearing inoculant, an Orthomyxoviridae bearing inoculant, a Paramyxoviridae bearing inoculant, a Togaviridae bearing inoculant, a Picornaviridae bearing inoculant, a Coronaviridae bearing inoculant, an Adenoviridae bearing inoculant, a Poxviridae bearing inoculant, a Hepadnaviridae bearing inoculant, a Reoviridae bearing inoculant, a Parvoviridae (including a Parvovirinae and/or Densovirinae bearing inoculant), a Rhabdoviridae bearing inoculant, a Bunyaviridae bearing inoculant, a Bromoviridae bearing inoculant, a Totiviridae bearing inoculant, a Tectiviridae bearing inoculant, a Plasmaviridae bearing inoculant, a Myoviridae bearing inoculant, a Siphoviridae bearing inoculant, a Podoviridae bearing inoculant, a Rudiviridae bearing inoculant, a bearing inoculant, a Corticoviridae bearing inoculant, a Lipothrixviridae bearing inoculant, a Plasmaviridae bearing inoculant, a Fuselloviridae bearing inoculant, a Phycodnaviridae bearing inoculant, an Iridoviridae bearing inoculant, a Polydnaviridae bearing inoculant, a Polyomaviridae bearing inoculant, a Papillomaviridae bearing inoculant, a bearing inoculant, an Ascoviridae bearing inoculant, a Baculoviridae bearing inoculant, a Nimaviridae bearing inoculant, an Asfarviridae bearing inoculant, an Inoviridae bearing inoculant, a Microviridae bearing inoculant, a Geminiviridae bearing inoculant, a Circoviridae bearing inoculant, a Nanoviridae bearing inoculant, a Pseudoviridae bearing inoculant, a Metaviridae bearing inoculant, a Caulimoviridae bearing inoculant, a Cystoviridae bearing inoculant, a Birnaviridae bearing inoculant, a Totiviridae bearing inoculant, a Chrysoviridae bearing inoculant, a Partitiviridae bearing inoculant, a Hypoviridae bearing inoculant, a Filoviridae bearing inoculant, a Bornaviridae bearing inoculant, an Arenaviridae bearing inoculant, a Leviviridae bearing inoculant, a Dicistroviridae bearing inoculant, a Sequiviridae bearing inoculant, a Comoviridae bearing inoculant, a Potyviridae bearing inoculant, a Caliciviridae bearing inoculant, an Astroviridae bearing inoculant, a Nodaviridae bearing inoculant, a Tetraviridae bearing inoculant, a Tombusviridae bearing inoculant, an Arteriviridae bearing inoculant, a Roniviridae bearing inoculant, a Bromoviridae bearing inoculant, a Closteroviridae bearing inoculant, a Barnaviridae bearing inoculant, a Luteoviridae bearing inoculant, a Narnaviridae bearing inoculant, a Pospiviroidae bearing inoculant, an Avsunviroidae bearing inoculant, and/or a prion bearing inoculant.

In a sub-embodiment of the present invention, the viral inoculant is a Retroviridae bearing inoculant. In a particular embodiment of the invention, the viral inoculant is an HIV-bearing inoculant, wherein the HIV includes the many clades, types and subtypes of HIV. In a particular embodiment of the invention, the viral inoculant is an HIV-1 bearing inoculant (Clade A, B, C, D, F, H, and/or O) and/or HIV-2 (Clade A and/or B) bearing inoculant. In another particular embodiment of the invention, the viral inoculant is a Human T-lymphotropic virus 2 (HTLV-2) bearing inoculant.

In a sub-embodiment of the present invention, the viral inoculant is a Herpesviridae bearing inoculant. In a particular embodiment of the invention, the viral inoculant is an HSV-1 or HSV-2 bearing inoculant. In another particular embodiment of the present invention, the viral inoculant is a human herpesvirus 3 (varicella-zoster virus) bearing inoculant. In yet another particular embodiment of the present invention, the viral inoculant is a CMV-bearing inoculant. In a still another particular embodiment of the present invention, the viral inoculant is an EBV-bearing inoculant. In a still another particular embodiment of the present invention, the viral inoculant is a human herpesvirus 6-bearing inoculant. In a still another particular embodiment of the present invention, the viral inoculant is a human herpesvirus 7-bearing inoculant. In a still another particular embodiment of the present invention, the viral inoculant is a human herpesvirus 8 (Kaposi's sarcoma-associated herpesvirus)-bearing inoculant.

In another sub-embodiment of the present invention, the viral inoculant is a Flaviviridae bearing inoculant. In a particular embodiment of the invention, the viral inoculant is a flavivirus bearing inoculant, wherein the flavivirus is, for example, a Dengue virus (Dengue virus, Dengue virus type 1, Dengue virus type 2, Dengue virus type 3, Dengue virus type 4), a Japanese encephalitis virus (Alfuy Virus, Japanese encephalitis virus, Kookaburra virus, Koutango virus, Kunjin virus, Murray Valley encephalitis virus, St. Louis encephalitis virus, Stratford virus, Usutu virus, West Nile Virus), a Modoc virus, a Rio Bravo virus (Apoi virus, Rio Brovo virus, Saboya virus), a Ntaya virus, a Tick-Borne encephalitis (tick born encephalitis virus), a Tyuleniy virus, an Uganda S virus and/or a Yellow Fever virus. In another particular embodiment of the invention, the viral inoculant is a hepacivirus bearing inoculant, wherein the hepacivirus is, for example, a hepatitis C virus (HCV) and/or its clades, type or subtype. In yet another particular embodiment of the invention, the viral inoculant is a pestivirus bearing inoculant, wherein the pestivirus is, for example, Bovine Viral Diarrhea Virus-2 (BVDV-2), Pestivirus type 1 (including BVDV), Pestivirus type 2 (including Hog Cholera Virus) and/or Pestivirus type 3 (including Border Disease Virus).

In yet another sub-embodiment of the present invention, the viral inoculant is a Orthomyxoviridae bearing inoculant. In a particular embodiment of the invention, the viral inoculant is an Influenzavirus A bearing inoculant. In another particular embodiment of the invention, the viral inoculant is an Influenzavirus B bearing inoculant. In yet another particular embodiment of the invention, the viral inoculant is an Influenzavirus C bearing inoculant. In yet another particular embodiment of the invention, the viral inoculant is an Influenzavirus D bearing inoculant.

In yet another sub-embodiment of the present invention, the viral inoculant is a Paramyxoviridae bearing inoculant. In a particular embodiment of the invention, the viral inoculant is a Paramyxovirnae bearing inoculant. In an even more particular embodiment of the invention, the viral inoculant is a paramyxovirus, wherein the paramyxovirus is, for example, a Sendai virus, such as human parainfluenza virus 1 and human parainfluenza virus 3. In an even more particular embodiment of the invention, the viral inoculant is a human parainfluenza virus 1 bearing inoculant. In another even more particular embodiment of the invention, the viral inoculant is a human parainfluenza virus 3 bearing inoculant. In another particular embodiment of the invention, the viral inoculant is a rubulavirus bearing inoculant. In an even more particular embodiment of the invention, the viral inoculant is a human parainfluenza virus 2 bearing inoculant. In another even more particular embodiment of the invention, the viral inoculant is a human parainfluenza virus 4 bearing inoculant. In an even more particular embodiment of the invention, the viral inoculant is a mumps virus bearing inoculant. In another particular embodiment of the invention, the viral inoculant is a morbillivurs bearing inoculant. In more particular embodiment of the invention, the viral inoculant is a measles virus bearing inoculant. In another particular embodiment of the invention, the viral inoculant is a Pneumovimae bearing inoculant.

In a more particular embodiment of the invention, the viral inoculant is a respiratory syncytial virus (RSV) bearing inoculant.

In yet another sub-embodiment of the present invention, the viral inoculant is a Coronaviridae bearing inoculant. In a particular embodiment of the invention, the viral inoculant is a human respiratory coronavirus (HCV-229E) bearing inoculant. In another particular embodiment of the invention, the viral inoculant is a human respiratory coronavirus (HCV-OC43) bearing inoculant. In yet another particular embodiment of the invention, the viral inoculant is a torovirus bearing inoculant, such as a human torovirus bearing inoculant.

In yet another sub-embodiment of the present invention, the viral inoculant is a Togaviridae bearing inoculant. In a particular embodiment of the invention, the viral inoculant is an alphavirus bearing inoculant. In another particular embodiment of the invention, the viral inoculant is a rubivirus bearing inoculant. In an even more particular embodiment of the invention, the viral inoculant is a Rubella virus bearing inoculant. In another even more particular embodiment of the invention, the viral inoculant is a Sindbis virus bearing inoculant. In another even more particular embodiment of the invention, the viral inoculant is EasternmWestem encephalitis virus bearing inoculant.

In yet another sub-embodiment of the present invention, the viral inoculant is a Picornaviridae bearing inoculant. In a particular embodiment of the invention, the viral inoculant is a human rhinovirus bearing inoculant, wherein the human rhinoviruses can be any one of the at least 105 serotypes (a classification scheme based on the variation of surface epitopes), which represent the most common etiological agent for the common cold. In a particular embodiment of the invention, the viral inoculant is an enterovirus. In an even more particular embodiment of the invention, the viral inoculant is a Human polioviruses 1, 2, and 3 (A23-echovirus; echo=Enteric Cytopathic Human Orphan viruses ) (3 serotypes), Human coxsackieviruses A1-22, 24 (23 serotypes), Human coxsackieviruses B1-6 (swine vesicular disease virus is very similar to coxsackie B5 virus) (6 serotypes), Human echoviruses 1-7, 9, 11-27, 29-34 (30 serotypes; these viruses show a seasonal, epidemic pattern of infection primarily associated with meningitis, paralysis (usually less severe than acute poliomyelitis), and myocarditis), Human enteroviruses 68-71 (4 serotypes), and/or Vilyuisk virus (1 serotype) bearing inoculant. In a particular embodiment of the invention, the viral inoculant is cardiovirus bearing inoculant, wherein the cardiovirus can be, for example, an encephalomyocarditis (EMC) virus (a mouse virus that can infect humans, elephants, and squirrels; includes mengovirus, Maus-Elberfield virus, and the Columbia virus) and Theiler's murine encephalocyelitis (TME) virus (TO, GDVII). In another particular embodiment of the invention, the viral inoculant is hepatovirus bearing inoculant. In yet another particular embodiment of the invention, the viral inoculant is human hepatitis virus A bearing inoculant. In yet another particular embodiment of the invention, the viral inoculant is a severe acute respiratory syndrome (SARS) Co-V bearing inoculant.

In yet another sub-embodiment of the present invention, the viral inoculant is a Hepadnaviridae bearing inoculant. In a particular embodiment of the invention, the viral inoculant is a human hepatitis B virus (HBV) bearing inoculant.

In yet another sub-embodiment of the present invention, the viral inoculant is a Adenoviridae bearing inoculant. In a particular embodiment of the invention, the viral inoculant is a human adenovirus A, B, C, D, E, and/or F bearing inoculant.

In yet another sub-embodiment of the present invention, the viral inoculant is a Arenaviridae bearing inoculant. In a particular embodiment of the invention, the viral inoculant is a human hepatitis D virus (HDV) bearing inoculant.

In yet another sub-embodiment of the present invention, the viral inoculant is a Caliciviridae bearing inoculant. In a particular embodiment of the invention, the viral inoculant is a human hepatitis E virus bearing inoculant.

In yet another sub-embodiment of the present invention, the viral inoculant is a prion bearing inoculant. In a particular embodiment of the invention, the inoculant is a prion bearing inoculant, wherein the prion is the causative agent of a spongiform encephalopathy such as Scrapie, Bovine spongiform encephalopathy (BSE), mad cow disease, Kuru, Creutzfeldt-Jakob disease (CJD), Gerstmann-Straussler-Scheinker syndrome (GSS), and/or Fatal familial insomnia (FFI).

In one embodiment of the present invention, the inoculant is a bacteria-bearing inoculant. In a particular embodiment of the present invention, the inoculant is a Gram-positive bacteria-bearing inoculant. In another embodiment of the invention, the inoculant is a Gram-negative bacteria-bearing inoculant. Non-limiting examples of inoculants include the blood, plasma or serum of a person infected with bacteria, a bacterial lysate, tissue from a person infected with a bacteria, lysates of cysts or other inclusion bodies containing bacteria, a purified bacterial preparation grown in vitro, or a suspension of bacteria in saline, plasma, or another biological fluid.

In one embodiment, the inoculant is blood, plasma or serum derived from a mammal, such as a human, infected with a virus. The virus may be, for example, HIV, HBV, HCV, a respiratory virus (e.g., influenza A, influenza B, RSV or SARS) or a herpes virus. In one embodiment, the virus is HIV. In a particular embodiment, the inoculant is plasma derived from a mammal, such as a human, infected with HIV. Non-limiting examples of HIV-bearing inoculants include the blood, plasma or serum of a mammal (e.g., a human) infected with HIV, a HIV viral lysate, purified HIV or naturally occurring or synthetic HIV viral proteins or peptides (glycosylated or unglycosylated). In a particular embodiment, the inoculant is plasma derived from an HIV positive individual. In one embodiment, the HIV positive blood, plasma or serum used as the inoculant come from an HIV positive individual with a viral load greater than 2,000. Plasma from an HIV positive individual manifesting clinical symptoms of HIV can be used as the inoculant.

In one embodiment, attenuated or heat treated virus can be used.

In a particular embodiment of the invention, the inoculant contains two or more different immunogens. In a particular embodiment of the invention, inoculant contains two or more viral immunogens. In one embodiment, one of these two or more viral immunogens is derived from HIV. In another embodiment, the inoculant comprises a viral immunogen and a bacterial immunogen. In a particular embodiment, the inoculant contains a HIV immunogen in combination with a Gram-positive bacteria-bearing immunogen.

According to one embodiment of the present invention, human blood is drawn from a virus positive patient using standard, sterile, phlebotic techniques. Preferably, the viral positive donors are between 18 and 65 years of age. Preferably, donors should appear healthy, not be under the influence of drugs or alcohol, and weigh in excess of 50 kg (110 lb). The following criteria of health can also be useful: body temperature less than 37.5° C.; pulse regular (50 to 100 beats per minute); blood pressure lower than 180 mm Hg systolic and 100 mm Hg diastolic; hemoglobin greater than 12.5 g/l and hematocrit greater than 38%. The blood is then processed to produce plasma according to techniques well known to those skilled in the art.

The plasma obtained from a virus-positive, for example, an HIV-positive individual, can then be used to inoculate a mammal, such as a goat. The plasma can be injected one or more times. In addition, various adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and corynebacterium parvum. Such adjuvants are also well known in the art. Additional non-limiting examples of adjuvants suitable for use in the present invention are discussed further below.

The plasma and/or adjuvant can be injected in the mammal by one or more subcutaneous or intraperitoneal injections, though they can also be given intramuscularly, and/or intravenously.

The mammal can be given a sedative, for example Rompun, to facilitate handling of the mammal if necessary.

In one embodiment, at least 1 cc of human plasma is administered to the mammal. Preferably, between 1-10 cc of the plasma can be administered to the animal subcutaneously. Alternatively, at least 1, 2, 5, 7, 10, 15, 20, 25, 30, 40 or 50 cc of plasma is administered subcutaneously, intraperitoneally intramuscularly, and/or intravenously.

In a particular embodiment, the animal is inoculated and then re-inoculated after a period of time ranging from 1, 2, 3, 4, 5, 6, 7, 8, 9, 10, 11, 12 or more weeks. The second inoculation is also known as a booster.

In one embodiment of the invention, an inoculant is not used. Rather, the serum or plasma cocktail is prepared by obtaining plasma or serum from an animal, without prior inoculation.

(iii) Monitoring of Animal

The date of injection and the animal should preferably be monitored over a time period, ranging from at least 1 to at least 8 weeks. Blood samples can be obtained from the animal during this time and processed to provide high molecular-weight protein or biological agent depleted plasma or serum fractions (e.g., an immunoglobulin-depleted protein fraction). As described further below under section (iv) below, the samples can be tested to confirm the presence of one or more biological agents which exhibit anti-viral activity. For example, sample can be measured for the ability of the plasma to inhibit HIV infection of CD4 cells in vitro. The mammal can be a goat. Once anti-viral activity is observed in the fraction, the serum should be obtained. It is known that in goats three weeks is the standard period of incubation for generating a sufficient immune response

(iv) Removal of Blood from Innoculated Animal

To obtain the plasma or serum from the mammal, blood has to be collected. Any means to do this which accomplishes the desired goal is suitable. It is preferable to obtain large quantities of blood from the mammal, for example 10-30 cc of blood from a rabbit or similar sized animal and higher quantities from larger animals. The blood should begin to flow immediately through the tubing to the syringe, vacutainer, or open tube/bottle. If a syringe is used, gently draw on the syringe to collect the blood, and once the syringe is full, change syringes by disconnecting from the infusion set or needle hub. (See, for example, McGuill, M. W. and Rowan, A. N., “Biological Effects of Blood Loss: Implications for Sampling Volumes and Techniques,” ILAR News, Vol. 31(4), Fall 1989, pp 5-20).

Whole blood is a circulating tissue composed of fluid plasma and cells (i.e., red blood cells, white blood cells and platelets). Plasma is that portion of blood that remains when the cells are removed. Serum is that portion of whole blood that remains when cells and the clotting proteins are removed. In one embodiment of the invention, the blood is collected in a manner that prevents coagulation in order to obtain plasma not serum. A variety of methods are know in the art for preventing coagulation of drawn blood, including without limitation, collecting the blood in tubes or other types of collecting means that have been treated with an anticoagulant. Anticoagulant coated test tubes of this type are widely available commercially. Suitable anticoagulants include, but are not limited to, EDTA, heparin, citrate or oxalate. Tube inversions allow proper mixing of anticoagulant additives and blood. Alternatively, a syringe and the infusion set tubing used in harvesting the blood can be filled with an anticoagulant prior to aid in the harvesting of blood. Alternatively, blood can be collected in a vacutainer or bottle that has been treated with an anticoagulant. Alternatively, anticoagulants can be added to the plasma component of blood after the cellular elements have been removed, for example by centrifugation as described below.

The animal can be sedated. For example, 0.5 cc Rompun can be used to sedate, for example, a goat. In another example, Torbugesic (butorphanol; 1 mg/kg) and acepromazine maleate (1 mg/kg) can be used to sedate, for example, a rabbit. After the animal is sedated, the blood can be collected. One way to remove blood from an animal is to cannulate an artery, for example the external jugular artery. The mammal can be a goat and for a goat, at least 18 gauge needle can be used to extract at least 150 cc of blood, preferable between 200-400 cc of blood. In another example, a needle, at least 21 gauge, is connected to an infusion device, such as an E-Z infusion set, to a syringe, for example, at least a 10 or 20 cc syringe.

As the blood is collected, it is preferably stored in a cooled enviromnent, for example on ice or in a refrigerator or freezer.

(v) Treatment of Conditioned Blood to Form Plasma or Serum

The following description describes one way in which the blood can be treated to form serum.

Cellular elements are separated from blood by centrifugation. Centrifugation speeds and times are known to those skilled in the art, and may depend, for example, upon the type of tube used for blood collection. The specific gravity ranges for red cells are sufficiently different to enable isolation by centrifugation. Plasma is then obtained from the appropriate fragment. The plasma can be repeatedly centrifuged to minimize the number of residual cells in the plasma fraction. To obtain serum, the plasma is permitted to clot. Serum is then separated from clotted blood by centrifugation. Certain types of tubes known to those in the art may facilitate the separation process. For example, tubes containing a gel substance such that when the tube is centrifuged the cells go below the gel while the serum remains above.

(vi) Treatment of Plasma or Serum to Form a High Molecular Weight Protein or Biological Agent-Depleted Fraction

The anti-viral activity of the plasma or serum is then isolated from one or more of the various high molecular weight proteins (e.g., immunoglobulins, albumin) or biological agents present in serum or plasma. Plasma contains a mixture of hundreds of different kinds of proteins. (For a review, see Turner, M. W., and Hulme, B. (1970) The Plasma Proteins: An Introduction, Pitman Medical & Scientific Publishing Co., Ltd., London). Serum differs from plasma in that the clotting proteins (i.e., fibronectin) have been removed. The protein content of serum is approximately 60-80 mg/ml). The majority of serum protein is represented by a few, very abundant high molecular weight (HMW) proteins. Common high molecular weight proteins include, for example, immunoglobulins, albumin, transferrin, haptoglobin and lipoproteins.

Immunoglobulins (antibodies) are globular glycoproteins found in body fluids such as serum or on B cells where they act as antigen receptors. Immunoglobulins represent 10-25% of all serum proteins. They range in molecular weight from approximately 150,000-970,000 daltons. The five major classes of immunoglobulins (IgA, IgG, IgM, IgD and IgE), are distinguished by differences in the C regions of H chains of the molecule. They differ in size, charge, amino acid composition and carbohydrate content.

IgG is the dominant immunoglobulin (70-75%) in extracellular fluids like serum and has a molecular weight of approximately 150,000 daltons. IgM is the largest immunoglobulin, and has a molecular weight of 900,000 daltons. It represents approximately 10% of the total immunoglobulin pool. IgA concentrates in body fluids such as tears, saliva, and the secretions of the respiratory and gastrointestinal tracts. IgD accounts for less than 1% of the plasma immunoglobulins, and is almost exclusively found inserted into the membrane of B cells. IgE is normally present in only trace amounts, but it is responsible for the symptoms of allergy.

Albumin is a highly-water soluble protein with a molecular weight of approximately 66,000 Da. (For a review, see Peters T., Jr. All about Albumin: Biochemistry, Genetics, and Medical Applications Academic Press, San Diego, 1996) It is the most abundant protein in human blood, representing more than 55% of total serum proteins. It plays a role in the osmotic pressure of the plasma, and also functions as a carrier for hormones, enzymes, fatty acids, and metal ions. The average concentration of albumin in human serum is 4.0-4.8 g/100ml.

Transferrin is a metal-binding glycoprotein with a molecular weight of approximately 80,000 daltons. (For a review, see Huebers H A and Finch C A. Physiological Reviews (1987) 67: 520). The primary function of transferring is the transport of iron in plasma. It is also known as siderophilin.

Haptoglobin is a 100,000 dalton glycoprotein. It removes free hemoglobin from the circulation of vertebrates which binds free hemoglobin, preventing loss in the urine. Other diverse properties of human haptoglobin have been observed (see, e.g., Oh S K et al. J. Leuko. Biol., (1990) 47: 142-148; Cid M C et al. J. Clin. Invest. (1993) 91: 977-985).

Lipoproteins are lipid-protein complexes which permit the transport of otherwise insoluble lipids through the blood stream. The major serum lipoproteins include chylomicrons, very low density lipoproteins (VLDL), low density lipoproteins (LDL), intermediate-density lipoproteins (IDL), and high-density lipoproteins (HDL).

Low molecular weight proteins found in plasma and serum include cytokines, chemokines, peptide hormones, as well as proteolytic fragments of large proteins. Ctyokines and growth factors are typically between 6 and 50 kD, and more commonly between 10 and 30 kD. The molecular weight of various low molecular weight proteins commonly found in human serum is detailed in the commonly available BioSource catalog.

Proteins can be separated from plasma or serum by fractionation. Fractionation strategies can vary in specificity, from very general to highly specific for a particular activity of interest. Fraction methods can be used alone or in combination. The goal of fractionation is to obtain a fraction enriched for an activity of interest. In the present invention, the activity of interest is believed to reside in a fraction of plasma or serum depleted of one or more high molecular weight proteins such as albumins and immunoglobulins. Put another way, the activity of interest is found in the low molecular weight fraction.

In a particular embodiment of the present invention, the desired product of fractionation is a low molecular weight protein fraction enriched for proteins and/or other biological agents between approximately 6 and approximately 50 kD

In a particular embodiment of the present invention, the desired product of fractionation is a low molecular weight protein fraction enriched for proteins and/or other biological agents between approximately 6 and approximately 30 kD.

In a particular embodiment of the present invention, the desired product of fractionation is a low molecular weight protein fraction enriched for proteins and/or other biological agents between approximately 6 and approximately 20 kD.

In a particular embodiment of the present invention, the desired product of fractionation is a low molecular weight protein fraction enriched for proteins and/or other biological agents between approximately 6 and approximately 14 kD.

In a particular embodiment of the present invention, the desired product of fractionation is a low molecular weight protein fraction enriched for proteins and/or biological agents between approximately 6 and approximately 10 kD.

In a particular embodiment of the present invention, the desired product of fractionation is a low molecular weight protein fraction enriched for proteins and/or biological agents between approximately 30 and approximately 50 kD.

In a particular embodiment of the present invention, the desired product of fractionation is a low molecular weight protein fraction enriched for proteins and/or other biological agents between approximately 30 and approximately 40 kD.

In a particular embodiment of the present invention, the desired product of fractionation is a low molecular weight protein fraction enriched for proteins and/or other biological agents between approximately 15 and approximately 25 kD.

In a particular embodiment of the present invention, the desired product of fractionation is a low molecular weight protein fraction enriched for proteins and/or other biological agents between approximately 20 and approximately 25 kD.

In one embodiment of the present invention, the desired product of fractionation is a low molecular weight protein fraction enriched for proteins and/or other biological agents with a molecular weight of approximately 12.2 kD.

In another embodiment of the present invention, the desired product of fractionation is a low molecular weight protein fraction enriched for proteins and/or other biological agents with a molecular weight of approximately 14.1 kD.

In a further embodiment of the present invention, the desired product of fractionation is a low molecular weight protein fraction enriched for proteins and/or other biological agents with a molecular weight of approximately 28.6 kD

In yet another embodiment of the present invention, the desired product of fractionation is a low molecular weight protein fraction enriched for proteins and/or other biological agents with a molecular weight of approximately 29 kD.

In yet a further embodiment of the present invention, the desired product of fractionation is a low molecular weight protein fraction enriched for proteins and/or other biological agents with a molecular weight of approximately 30.1 kD.

In a particular embodiment of the present invention, the desired product of fractionation is a low molecular weight protein fraction enriched for proteins and/or other biological agents with a molecular weight of approximately 49.4 kD.

In another embodiment of the present invention, the desired product of fractionation is a low molecular weight protein fraction enriched for proteins and/or other biological agents with a molecular weight of approximately 53 kD.

It may require multiple fractionation steps to isolate the anti-viral activity from one or more of the high-molecular weight proteins. Specifically, the anti-viral activity may initially fractionate with the high molecular weight proteins, which high molecular weight fraction must then be further processed to isolate the anti-viral activity from the high molecular weigh proteins. Alternatively, the anti-viral activity may fractionate from the high molecular weight protein or proteins in a single fractionation step.

A wide variety of methods are available to fractionate plasma or serum to isolate proteins. Such methods can be broadly divided into those which divide the protein between two phases (e.g., a solid and liquid) and those which separate proteins by different rates of movement through a material, such as a chromatographic column or electrophoresis gel. Any method capable of achieving the desired result is considered suitable for use in the present invention. These methods can be used alone, or in combination. Fractionation can involve a single step, or multiple steps. When multiple fractionation steps are used, the fractionation method may be the same or different.

Fractional precipitation. Fractional precipitation can be used to isolate the anti-viral activity of the initial serum or plasma fraction from high molecular weight proteins, such as immunoglobulins and albumins. Non-limiting examples of fractional precipitation methods include solvent, salt, isolectric, hydrophilic polymer and heat precipitation. All fractional precipitation methods rely on bringing protein out of solution by altering the medium to reduce its solubility. Once insoluble, the protein can be separated form the mixture by centrifugation or filtration. Organic solvent precipitation methods are suitable for use in the method of the present invention. Addition of the solvent results in a decrease in the dielectric constant of the medium, which produces a decrease in protein solubility. Solvents may include, for example, 2 methyl-2,4-pentane diol (MPD), Dimethyl Sulfoxide (DMSO) and ethanol. In a particular embodiment, cold alcohol fractionation or ethanol fractionation, also known as the Cohn-Oncley method, is used (Cohn E J et al. J Am Chem Soc 1946; 68: 459-75). This method involves the precipitation of proteins under varying conditions of ethanol and pH conditions.

A variety of cold ethanol fractionation methods are known in the art for isolating albumin and immunoglobulin from plasma (See e.g., Cohn E J et al. J Am Chem Soc (1946) 68: 459-75; Hink J H et al. Vox Sang (1957) 2: 174-86; Kistler P et al. Vox Sang (1962) 7: 414-24). The Cohn and Kitler methods are compared in More J E et al. In: Harris J R, ed. Blood Separation & Plasma Fractionation. New York: Wiley, 1991; 261-306). Coagulation factors are removed as cryoprecipitate on initial thawing of the plasma before cold ethanol fractionation. With either method, an initial low ethanol precipitation stage removes the fibrinogen from the source plasma. Immunoglobulins are precipitated by raising the ethanol concentration to 25% at pH 6.9 for the Cohn method or 19% at pH 5.85 for the Kistler and Nitschmann method, while albumin remains in solution. Albumin is then isolated from the majority of the other plasma contaminants (mainly alpha and beta globulins), which are precipitated by the further addition of ethanol to a final ethanol concentration of 40%. This is carried out in two stages in the Cohn process but as a single step in the Kistler and Nitschmann method. In a final step, the albumin is itself precipitated near its isoelectric point. In an alternate approach to solvent precipitation, certain proteins in a mixture can be specifically inactivated and denatured by an organic solvent.

Proteins can also be separated from plasma or serum by salt precipitation. Protein solubility is a function of the physiochemical nature of the proteins, pH temperature and the concentration of the salt used. It also depends on whether the salt is Kosomtropic (stabilizes water structure) or Chaotropic (disrupts water structure). Many types of salts (e.g., ammonium sulfate) can be employed to effect protein separation and purification. Ammonium sulfate is common used because of it is highly soluble, relatively inexpensive and generally preserves protein function. Using the appropriate concentration range of the given salt, a protein of interest can be preferentially isolated from a protein mixture. According to this method, increasing amounts of ammonium sulfate are added to give a certain percentage saturated, followed by a period of time to permit proteins to precipitate, and a centrifugation step to collect the precipitate.

In one embodiment of the present invention, ammonium sulfate precipitation is used to isolate the anti-viral activity of the plasma or serum from one or more high-molecular weight proteins present in the unfractionated plasma or serum. In one embodiment, a single ammonium sulfate precipitation step is sufficient to isolate the anti-viral activity of the plasma or serum from one or more high-molecular weight proteins present in the unfractionated plasma or serum. In another embodiment, ammonium sulfate precipitation is used to in combination with one or more additional fractionation steps or methods, either the same or different, to isolate the anti-viral activity of the plasma or serum from one or more high-molecular weight proteins present in the unfractionated plasma or serum.

In a particular embodiment, ammonium sulfate precipitation is used to isolate the anti-viral activity of the plasma or serum from immunoglobulins present in the unfractionated plasma or serum. These immunoglobulins may include IgG, IgM or both IgG or IgM.

Hydrophilic polymers such as polyethylene glycol (PEG) can also be used to precipitate proteins according to the present invention. PEG varies in chain lengths from average mol wt 1000 to 40000. Those of higher molecular weight are frequently useful in concentration schemes, with the most common PEG6000.

Isoelectric precipitation can also be used to fractionate proteins in the present invention. In general, proteins are positively charged at a low pH and negatively charged at a high pH. A protein is the least soluble when the pH of the solution is at its isoelectric point, i.e., the pH at which a protein molecule has a net charge of zero.

Heat precipitation also permits isolation of proteins according to the present invention. This method is typically used to remove contaminating proteins from a protein-containing solution. The stability of different proteins at elevated temperature varies, and if the desired protein has a greater heat stability than contaminating proteins, incubation at elevated temperatures (e.g., 45-70° C.) for a period of (i.e., varying from a few minutes to a few hours) produces precipitation of the unwanted proteins.

Dialysis and ultrafiltration can also be used to provide low-resolution protein fractionation. In dialysis, the protein sample is enclosed in a bag consisting of a semipermeable membrane (made of cellulose) and exposed to a large volume of a desired buffer. The low-molecular weight compounds (buffering agents, salts) pass freely through the membrane pores whereas the protein is retained. In ultrafiltration methods, the pores are generally larger and allow smaller proteins to pass through. Ultrafiltration typically employs pressure to force the sample through. Membranes with various molecular weight cutoffs are commercially available (e.g., from less than 10 to more than 100 kDa). Centrifugal ultrafiltration can also be used to deplete serum of large, highly abundant proteins such as albumin. (Tirumalai RS et al. Molecular & Cellular Proteomics (2003) 2:1096-1103.

Chromatographic Fractionation. Chromatographic fractionation of plasma or serum can also be used the anti-viral activity from serum or plasma according to the present invention. In general, chromatography refers to any of a number of methods in which solutes are fractionated by partitioning between a mobile or buffer and an immobile or matrix, phase. Column chromatography is one type, and involves passing the starting material through a column which is constantly being washed through with a suitable buffer. As the protein enters the column, it interacts with the matrix of the column which can take many forms. Types of chromatography suitable for use in the present invention include, without limitation, gel filtration, ion exchange, and affinity chromatography, either alone or in combination with one or more additional fraction methods.

(i) Gel filtration chromatography, also known as molecular exclusion or gel permeation chromatography, separates molecules on the basis of size. In this method, the stationary phase is a gel matrix with a well-defined range of pore sizes is used. Large proteins do not enter the pores of the chromatographic matrix, but pass through, into the interstitial space between the matrix beads; this space is also known as the void volume, V0. These large proteins migrate more rapidly than small molecules which diffuse into and back out of the resin and consequently are partly trapped and fall behind. Proteins of intermediate size will penetrate to varying degrees into the beads and thus are separated from each other on the basis of their size. Low-pressure gel beads are capable of separating molecules from a molecular weight of a few hundred to multimeric proteins weighing in the millions range. These gel filtration resins are made from a variety of materials, including dextran, agarose, and polyacrylamide and are available in various pore sizes. Commercial gels include, for example, Bio-Gel (Bio-Rad) and Sephadex/Sepharose (Amersham Pharmacia Biotech).

In one embodiment of the present invention, gel filtration is used to isolate the anti-viral activity of the plasma or serum from one or more high-molecular weight proteins present in the unfractionated plasma or serum. High molecular weight proteins, including immunoglobulins and serum albumins, typically fractionate in the first eluted fractions to come off of the column. The lower molecular weight proteins, such as cytokines, typically come off of the column in the latter fractions.

In one embodiment, a single gel filtration step is used to isolate the anti-viral activity of the plasma or serum from one or more high-molecular weight proteins present in the unfractionated plasma or serum. In another embodiment, a gel filtration step is used to in combination with one or more additional fractionation steps or methods, either the same or different, to isolate the anti-viral activity of the plasma or serum from one or more high-molecular weight proteins present in the unfractionated plasma or serum.

(ii) Ion exchange chromatography involves the use of a stationary phase matrix with covalently linked anions or cations. Solute ions of the opposite charge in the mobile liquid phase are attracted to the resin by electrostatic forces. Under specific starting conditions of buffer, pH, and ionic strength, the net charge on the protein of interest can be manipulated to interact with the matrix. These conditions are well known to those in the art. IEC media are available in differing charges, pore sizes, and support strengths (i.e., low-pressure to high-pressure tolerant). Commercial sources of IEC media include Amersham-Pharmacia, Bio-Rad, Dionex, Hewlett Packard, Merck, Perseptive Biosystems, and TosoHaas, among others.

In one embodiment of the present invention, the anti-viral activity is isolated from one or more high molecular weight proteins present in the initial plasma or serum sample using ion exchange chromatography. In one embodiment, the anti-viral activity is isolated from one or more high molecular weight proteins present in the initial plasma or serum sample using a single ion exchange chromatography step. In another embodiment, the anti-viral activity is isolated from one or more high molecular weight proteins present in the initial plasma or serum sample using a ion exchange chromatography step in combination with one or more additional fractionation steps or methods, either the same or different.

In a particular embodiment, the anti-viral activity is isolated from immunoglobulins (i.e., IgG, IgM or both) using ion exchange chromatography. In another embodiment, anti-viral activity is isolated from two or more high molecular weight proteins present in the initial serum sample. In a particular embodiment, the anti-viral activity is isolated from immunoglobulins and albumin.

(iii) Affinity chromatography involves the specific interaction between one molecule in the sample and a second molecule immobilized on a stationary phase. Proteins can be used to isolate antibodies and vice versa. The affinity may be to a specific protein or a group of proteins. If the protein to be isolated is a gamma globulin, protein A is often used. Serum which contains the secreted antibodies is put through the affinity column, and the antibodies bind to the protein A attached to the column gel. Other ligands suitable for use in isolating components of blood include heparin (clotting-factor proteins), lectins (glycoproteins), antibodies (unique antigens), and enzyme inhibitors or cofactors (enzymes). For example, VLDL and LDL can be removed from a sample using antibody-based affinity chromatography (also known as immunoabsorption). Sources include, for example, Amersham-Pharmacia and Bio-Rad.

In a particular embodiment of the present invention, the anti-viral activity is isolated from one or more high-molecular weight proteins in the initial serum or plasma sample using affinity chromatography. Affinity chromatography may be used alone or in combination with other fractionation steps or methods detailed herein. In a particular embodiment, a protein G affinity column is used to deplete the sample of IgG to further isolate the anti-viral activity.

Methods of isolating albumin from serum involving chromatographic adsorbents and immunoaffinity methods have been reported (SatoAK et al., Biotechnol. Prog. (2002) 18, 182-192; Dockal M et al. J. Biol. Chem. (1999) 274, 29303-29310; Rothemund D et al. Proteomics (2003) 3, 279-287).

Recent advances in the study of the human proteome have led to the development of techniques to remove high abundance proteins from serum in order to isolate LMW proteins (see generally, Tirumalai RS et al. Molecular & Cellular Proteomics (2003) 2:1096-1103). Several of these methods are designed to retain LMW proteins which would otherwise be lost in the fractionation because they tend to bind to HMW proteins. These methods are considered suitable for use in isolating the anti-viral activity of the plasma or serum of a mammal exposed to an inoculant (e.g., HIV) from the high molecular weight proteins present therein, including, for example, immunoglobulins and albumins.

Commercial affinity depletion products are available to separate albumin and immunoglobulins from serum or plasma. For example, the ProteoExtract™ Albumin/IgG Removal Kit (CalbioChem) provides highly specific and efficient depletion of albumin and IgG from plasma or serum. Depletion of albumin and IgG removes up to 75% of total serum proteins. Applied BioSystems also manufactures affinity depletion cartridges for the removal of albumin (POROS® Anti-HAS support) and IgG (POROS® Protein G cartridge) from serum (Application Note: Protemics. Affinity Depletion Cartridges for Removal of Human Serum Albumin and Immunoglobulins from Human Serum. Applied Biosystems). Using these products, greater than 99% of IgG and albumin can be removed from serum. These commercial affinity depletion products are considered suitable for use in preparing the plasma or serum fraction of the present invention.

Polyclonal antibodies can be used to deplete plasma or serum of high molecular proteins in a single step, including albumin, IgG, IgA, haptoglobin, transferrin, and antitrypsin, using liquid chromatography. Commercial sources of multi affinity removal systems include, for example, Agilent. This technique removes 85-90% of the high abundance proteins from serum. These multi-affinity removal systems are considered suitable for use in preparing the plasma or serum fraction of the present invention.

In one embodiment of the present invention, a single fractionation is sufficient to isolate the anti-viral activity of the plasma or serum from one or more high-molecular weight proteins such as albumins and immunoglobulins. Alternatively, two or more fractionation steps can be combined. Each step may be the same basic technique (e.g., multiple ammonium sulfate precipitations) or different (e.g., precipitation with cold ethanol in combination with chromatography or heat precipitation). Any method or combination of methods suitable for isolating the anti-viral activity in the initial plasma or serum sample from immunoglobulins and other high molecular weight proteins is considered suitable for use in the present invention.

In a particular embodiment, the anti-viral activity is isolated from immunoglobulins and albumin present in the initial plasma or serum sample by sequential ammonium sulfate precipitation in combination with DEAE-Blue column chromatography.

According to one embodiment of the present invention, proteins or biological agents with a molecular weight of greater than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 67, 68, 69, 70, 71, 72, 73, 74, 75, 76, 77, 78, 79, 80, 81, 82, 83, 84, 85, 86, 87, 88. 89, 90, 91, 92, 93, 94, 95, 96, 97, 98, 99, 100, 101, 102, 103, 104, 105, 106, 107, 108 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 or 145 kD are depleted from the initial serum or plasma sample to facilitate isolation of the anti-viral activity.

In a particular embodiment, the serum or plasma fraction is depleted of proteins or other biological agents with a molecular weight greater than approximately 10 kD to facilitate isolation of the anti-viral activity.

In a further embodiment, the serum or plasma fraction is depleted of proteins or other biological agents with a molecular weight greater than approximately 12.2 kD to facilitate isolation of the anti-viral activity.

In another embodiment, the serum or plasma fraction is depleted of proteins or other biological agents with a molecular weight greater than approximately 14.1 kD to facilitate isolation of the anti-viral activity.

In a particular embodiment, the serum or plasma fraction is depleted of proteins or other biological agents with a molecular weight greater than approximately 15 kD to facilitate isolation of the anti-viral activity.

In another embodiment, the serum or plasma fraction is depleted of proteins or other biological agents with a molecular weight greater than approximately 20 kD to facilitate isolation of the anti-viral activity.

In a further embodiment, the serum or plasma fraction is depleted of proteins or other biological agents with a molecular weight greater than approximately 25 kD to facilitate isolation of the anti-viral activity.

In another embodiment, the serum or plasma fraction is depleted of proteins or other biological agents with a molecular weight greater than approximately 28.6 kD to facilitate isolation of the anti-viral activity.

In yet another embodiment, the serum or plasma fraction is depleted of proteins or other biological agents with a molecular weight greater than approximately 29 kD to facilitate isolation of the anti-viral activity.

In one embodiment, the serum or plasma fraction is depleted of proteins or other biological agents with a molecular weight greater than approximately 30 kD to facilitate isolation of the anti-viral activity.

In a further embodiment, the serum or plasma fraction is depleted of proteins or other biological agents with a molecular weight greater than approximately 35 kD to facilitate isolation of the anti-viral activity.

In another embodiment, the serum or plasma fraction is depleted of proteins or other biological agents with a molecular weight greater than approximately 40 kD to facilitate isolation of the anti-viral activity.

In a further embodiment, the serum or plasma fraction is depleted of proteins or other biological agents with a molecular weight greater than approximately 45 kD to facilitate isolation of the anti-viral activity.

In another embodiment, the serum or plasma fraction is depleted of proteins or other biological agents with a molecular weight greater than approximately 49.1 kD to facilitate isolation of the anti-viral activity.

In a further embodiment, the serum or plasma fraction is depleted of proteins or other biological agents with a molecular weight greater than approximately 50 kD to facilitate isolation of the anti-viral activity.

In a further embodiment, the serum or plasma fraction is depleted of proteins or other biological agents with a molecular weight greater than approximately 53 kD to facilitate isolation of the anti-viral activity.

In another In a further embodiment, the serum or plasma fraction is depleted of proteins or other biological agents with a molecular weight greater than approximately 65 kD to facilitate isolation of the anti-viral activity.

In yet another embodiment, the serum or plasma fraction is depleted of proteins or other biological agents with a molecular weight of greater than approximately 90 kD to facilitate isolation of the anti-viral activity.

The term “depletion” is used to indicate a reduction in the amount of a compound(s) or molecule(s) (e.g., high molecular weights proteins) in a given sample after the sample is treated according to the method of the present invention. In one embodiment of the present invention, the sample is depleted of 100%, 99%, 98%, 97%, 96%, 95%, 94%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 56%, 54%, 53%, 52%, 51%, 50%, 49-45%, 44-40%, 39-35%, 34-30%, 29-20%, 19-10%, 10%-5%, 5%-1% of the high molecular weight proteins or biological agents such as an immunoglobulin, serum albumin, transferrin, haptoglobin or lipoproteins.

In one embodiment of the present invention, the plasma or serum is depleted of substantially all high molecular weight proteins or biological agents. In a particular embodiment, the plasma or serum is depleted of substantially all proteins with a molecular weight greater than approximately 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 or 145 kD.

As noted above, while the initial sample may be depleted of one or more high molecular weight proteins or biological agents to facilitate isolation of the anti-viral activity, the anti-viral activity may initially co-fractionate with one or more of the high molecular weight proteins or biological agents, such that the high molecular weight fraction must be further fractionated or processed to isolate the anti-viral activity present therein.

Transmission of infectious disease (i.e., by viruses, bacteria or parasites) remains a concern in the use of any blood or blood product such as plasma or serum. In a further embodiment of the present invention, the plasma or serum fraction can be sterilized prior to in vivo use. Any suitable method can be used to achieve sterilization as long as the method does not alter the product in such a way as to diminish its efficacy. Non-limiting examples of sterilization techniques suitable for use with the present invention include chemicals, heat, ultraviolet radiation and photosensitizing dyes. The plasma can also be filtered to achieve sterilization. Recent advances and new strategies for the inactivation and removal of infectious agents are contemplated for use in the present invention.

In a particular embodiment of the present invention, the plasma is repeatedly centrifuged and filtered. For example, the plasma is spun at approximately 32,000 rpm on a standard centrifuge. The resultant supernatant can then be transferred, preferably under sterile conditions using sterile techniques, and then suction filtered through a 0.5 micron filter. During this preparation, the sample can be kept on ice between the centrifugation and filtration steps. The plasma can then be passed over a filter, for example a filter with at least 0.2 micron pores, and then placed in an ultracentrifuge, preferably non-refrigerated, to spin at approximately 90,000 rpm for at least 20 minutes. The supernatant can then be placed in containers, preferable sterile, in an ultracentrifuge, preferably non-refrigerated, to spin at least 150,000 rpm for at least 20 minutes. After the centrifugation, the supernatant can be passed through an anhydrous filter. The plasma can be repeatedly filtered, preferably through a 0.2 micron filter and a smaller filter, such as a 0.1 micron filter. Passage through a 0.1 micron filter allows for the plasma to be deemed sterile.

(vi) Storage and Testing of Plasma or Serum Fraction

The resulting plasma or serum fraction can be placed in small aliquots (e.g., between 2-10 cc each) and stored for later use. Proper storage conditions for plasma and serum with respect to temperature and time are well known to those skilled in the art. For example, test tubes containing small aliquots of the plasma or serum fraction can be stored at −70° C., for at least 48 hours.

After a suitable time has passed for the samples to be stored, such as 48 hours, individual aliquots can be brought to room temperature for sterility testing. For example, the sample can be cultured under both anaerobic and aerobic conditions to test for contamination. If the cultures are negative, the remaining aliquots of can then be administered to a patient.

(vii) Administration of the Therapeutic Plasma or Serum Fraction to A patient in Need thereof

The plasma or serum fraction can be administered to a patient in need thereof through any means provided in this application (See Pharmaceutical Compositions below). The precise amount of the therapeutic fraction administered to a host or patient will be the responsibility of the attendant physician. However, the dose employed will depend on a number of factors, including the age and sex of the patient, the precise viral infection or related disorder being treated, and its severity. In one embodiment, the patient can receive a therapeutically effective dosage, preferably between 0.1 and 10 cc, most preferably 2-1 cc, if administered subcutaneously, and treatment duration can vary based on the severity of the HIV infection. For example, treatment can range from daily administration for a patient with a CD4 count of less than 400 to a single, unrepeated dose administered, for example, to an HIV positive still asymptomatic patient.

B. Agents That Can Be Used in Combination and/or Alternation With the Composition of the Present Invention

The plasma or serum fraction of the present invention can be administered alone or can be administered in combination or alternation with another agent that treats the viral infection or a related condition or concomitant opportunistic infection. In general, during alternation therapy, an effective dosage of each agent is administered serially, whereas in combination therapy, effective dosages of two or more agents are administered together. The dosages will depend on such factors as absorption, bio-distribution, metabolism and excretion rates for each drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. Examples of suitable dosage ranges can be found in the scientific literature and in the Physicians Desk Reference. Many examples of suitable dosage ranges for other compounds described herein are also found in public literature or can be identified using known procedures. These dosage ranges can be modified as desired to achieve a desired result.

The plasma or serum fraction of the present invention can be administered alone or in combination or alteranation with agent used to treat a viral infected caused by a virus listed in Section A above, including any virus listed as a suitable inoculant. Representative, non-limiting examples of anti-viral agents suitable for use in combination or alternation with the plasma or serum fraction of the present invention include anti-HIV agents, anti-HBV agents, anti-respiratory virus agents and anti-herpes virus agents.

(i) Anti-HIV Agents

In one embodiment, the plasma or serum fraction of the present invention can be administered alone or can be administered in combination or alternation with another anti-HIV agent or agent used to treat an HIV-related condition, opportunistic infection or co-infection.

The plasma or serum fraction of the present invention can be administered in combination or alternation with any anti-HIV agent or agent used to treat a related condition or co-infection. Other non-limiting examples of anti-HIV agents or agents used to treat related conditions that can be administered in combination or alternation with the plasma or serum fraction of the present invention for the treatment of HIV infections and related conditions include: Abacavir (Ziagen®; GlaxoSmithKline); Abacavir+Lamivudine (Epzicom®; GlaxoSmithKline); Abacavir+Zidovudine+Lamivudine (Trizivir®&; GlaxoSmithKline); Agenerase (Amprenavir; Glaxo Wellcome); Aldesleukin (Proleukin, Chiron Corp: Alitretinoin (Panretin, gel 0.1%, Ligand Pharmaceuticals); AMD11070 (Anormed Pharmaceuticals) Amphotericin B Liquid Complex (Abelcet, ABLC, Ambisome, The Liposome Company): Amprenavir (Agenerase®, GlaxoSmithKline); Atovaquone (Mepron, 566C80; Glaxo Wellcome Trimethoprim-Sulfamethoxazole (TMP-SMX); Atazanavir (Reyataz®; Bristol-Myers Squibb); Azithromycin (Zithromax; Pfizer, Inc.) Beta-L-Fd4C (Achillion Pharmaceuticals); BMS 561390 (Bristol-Myers Squibb); Calanolide A (Sarawak Medicam Pharmaceuticals); Capravirine (Pfizer and Agouron Pharmaceuticals); CCR5 Receptor Antagonist (Schering Plough); CD4-IgG2 (PRO 542, Progenics Pharmaceuticals) Cidofovir (Vistide, HPMPC; Gilead Sciences, Inc.); Clarithromycin (Biaxin, Klacid, Abbott Laboratories) Cytolin A (Amerimmune Pharmaceuticals); DAPD (Amdoxovir, Gilead Sciences); Daunorubicin-liposomal (DaunoXome, Nexstar); Delavirdine mesylate (DLV, Rescriptor, Pharmacia & Upjohn); Didanosine (ddI, Dideoxyinosine, Videx®, VidexEC®, Bristol Myers-Squibb); Doxorubicin hydrochloride-liposomal (Doxil, Sequus Pharmaceuticals, Inc.); Dronabinol (Marinol, Roxane Laboratories); Efavirenz (Sustiva®, DuPont Pharmaceuticals); Emtricitabine (Emtriva®, Gilead Sciences); Enfuvirtide (Fuzeon®, T 20, Roche and Trimeris); Erythropoietin (EPO, Epogen, Procrit, Amgen); Famciclovir (Famvir, SmithKline Beecham); Fluconazole (Diflucan, Pfizer, Inc.); Fomivirsen Sodium Injection (Vitravene intravitreal injectable, Isis Pharmaceuticals); Fosamprenavir (GW433908, VX-175, Lexiva®, Vertex and Glaxo Wellcome); Foscarnet (Foscavir, Astra Pharmaceuticals); Ganciclovir (IV, Oral)(Cytovene, DHPG, Syntex); Ganciclovir (Implant) (Vitrasert, Chiron Corporation); Gem 92 (Hybridon); GS-7340-02 (Gilead Sciences); Immune globulin (IV) (Gamimune N, Gamma Globulin, IGIV, Bayer Pharmaceutical Division); Indinavir sulfate (Crixivan®, IDV, MK-639, Merck & Co.); Interferon Alfa-2a (Roferon-A, Hoffmann-La Roche); Interferon Alfa2b (Intron-A, Schering-Plough); Interleukin-2 SA (Bay 50-4798, Bayer Pharmaceutical); Itraconazole (Sporanox, Janssen Pharmaceutical); Lamivudine (Epivir®, 3TC, Glaxo Wellcome); Lamivudine/Zidovudine (Combivir®, Glaxo Wellcome); Leukocyte interleukin injection (Multkine, Cel SCI); Lopinavir/Ritonavir (Kaletra®, Abbott Laboratories); Megestrol acetate (Megace, Ovarian, Mead Johnson Laboratories); Mycophenolate mofetil (CellCept, Hoffmann La Rouche Inc.); Nelfinavir mesylate (NFV, Viracept®, Agouron Pharmaceuticals); Nevirapine (Viramune®, BI-RG-587, Boehringer Ingelheim Pharmaceuticals, Inc.); Paclitaxel (Taxol, Bristol Myers-Squibb Pharmaceutical Research Institute); Pentamidine (aerosolized) (NebuPent, Fujisawa); PolyI-polyC12U (Ampligen, HemispheRX); Procaine HCL (Anticort, Samaritan Pharmaceuticals); Racivir (Pharmasset); REV 123 (Novartis Pharmaceuticals); Rifabutin (Ansamycin, Mycobutin, Adria Laboratories; Ritonavir (Norvir®, ABT-538, Abbott Laboratories Approved soft gelatin, 100 mg capsule); S-1360 (GW810781)(integrase inhibitor, GlaxoSmithKline); Saquinavir mesylate (Invirase [hard gel capsule], Fortovase (soft gel capsule), SQV, Hoffmann-La Roche; soft gel capsule); Somatropin rDNA (Serostim, Serono Laboratories); Stavudine (d4T, Zerit), Bristol Myers-Squibb; Sulfamethoxazole/Trimethoprim, (GlaxoWellcome)(Bactrim when combined with Trimethoprim; Septra when combined with Trimethoprim; SMX); Tenofovir disoproxil fumarate (Viread®, Gilead); Tenofovir+Emtricitabine (Truvada®, Gilead); Tipranavir (PNU-140690, Aptivus®, Boehringer Ingelheim); TMC125 (non-nucleoside reverse transcriptase inhibitor, Tibotec); Trimethoprim/Sulfamethoxazole, HoffmannLa-Roche(Bactrim when combined with Sulfamethoxazole; Septra when combined with Sulfamethoxazole; SMX); Trimetrexate glucuronate (with Leucovorin)(Neutrexin, TMTX, U.S. Bioscience); Trizivir (fixed-dose combination of Ziagen (abacavir/ABC), Retrovir (zidovudine/AZT), and Epivir (lamivudine/3TC)), Glaxo Wellcome; Valcyte (oral valganciclovir HCL, Roche Laboratories); Z-100 (Ancer 20,Zeria Pharmaceutical Company; Zalcitabine (ddC, Dideoxycytidine, HIVID®, Hoffmann-La Roche); Zidovudine (Azidothymidine, AZT, Retrovir®, ZDV. Glaxo Wellcome); and Zidovudine+Lamivudine (Combivir®), GlaxoSmithKline

A more comprehensive list of anti-HIV compounds that can be administered in combination and/or alternation with the composition of the present invention include (1S,4R)-4-[2-amino-6-cyclopropyl-amino)-9H-purin-9-yl]-2-cyclopentene-1-methanol succinate (“1592”, a carbovir analog; GlaxoWellcome); 3TC: (−)-β-L-2′,3′-dideoxy-3′-thiacytidine (GlaxoWellcome); a-APA R18893: a-nitro-anilino-phenylacetamide; A-77003; C2 symmetry-based protease inhibitor (Abbott); A-75925: C2 symmetry-based protease inhibitor (Abbott); AAP-BHAP: bishetero-arylpiperazine analog (Upjohn); ABT-538: C2 symmetry-based protease inhibitor (Abbott); AzddU:3′-azido-2′,3′-dideoxyuridine; AZT: 3′-azido-3′-deoxythymidine (GlaxoWellcome); AZT-p-ddI: 3′-azido-3′-deoxythymidilyl-(5′,5′)-2′,3′-dideoxyinosinic acid (Ivax); BHAP: bisheteroaryl-piperazine; BILA 1906: N-{1S-[[[3-[2S-{(1,1-dimethylethyl)amino]-carbonyl}-4R]-3-pyridinylmethyl)thio]-1-piperidinyl]-2R-hydroxy-1S-(phenylmethyl)-propyl]amino]-carbonyl]-2-methylpropyl}-2-quinolinecarboxamide (Bio Mega/Boehringer-Ingelheim); BILA 2185: N-(1,1-dimethylethyl)-1-[2S-[[2-2,6-dimethyl-phenoxy)-1-oxoethyl]amino]-2R-hydroxy-4-phenylbutyl]4R-pyridinylthio)-2-piperidine-carboxamide (BioMega/Boehringer-Ingelheim); BM+51.0836: thiazolo-iso-indolinone derivative; BMS 186,318: aminodiol derivative HIV-1 protease inhibitor (Bristol-Myers-Squibb); d4API: 9-[2,5-dihydro-5-(phosphonomethoxy)-2-furane1]adenine (Gilead); d4C: 2′,3′-didehydro-2′,3′-dideoxycytidine; d4T: 2′,3′-didehydro-3′-deoxythymidine (Bristol-Myers-Squibb); ddC; 2′,3′-dideoxycytidine (Roche); ddI: 2′,3′-dideoxyinosine (Bristol-Myers-Squibb); DMP-266: a 1,4-dihydro-2H-3,1-benzoxazin-2-one; DMP-450: {[4R-(4-a,5-a,6-b,7-b)]-hexahydro-5,6-bis(hydroxy)-1,3-bis(3-amino)phenyl]-methyl)-4,7-bis-(phenylmethyl)-2H-1,3-diazepin-2-one}-bismesylate (Avid); EBU-dM:5-ethyl-1-ethoxymethyl-6-(3,5-dimethylbenzyl)-uracil; E-EBU: 5-ethyl-1-ethoxymethyl-6-benzyluracil; DS: dextran sulfate; E-EPSeU:1-(ethoxymethyl)-(6-phenylselenyl)-5-ethyluracil; E-EPU: 1-(ethoxymethyl)-(6-phenyl-thio)-5-ethyluracil; FTC:β-2′,3′-dideoxy-5-fluoro-3′-thiacytidine (Triangle); HBY097:S-4-isopropoxy-carbonyl-6-methoxy-3-(methylthio-methyl)-3,4-dihydroquinoxalin-2(1H)-thione; HEPT: 1-[(2-hydroxyethoxy)methyl]-6-(phenylthio)thymine; HIV-1:human immunodeficiency virus type 1; JM2763: 1,1′-(1,3-propanediyl)-bis-1,4,8,11-tetraaza-cyclotetradecane (Johnson Matthey); JM3100:1,1′-[1,4-phenylenebis-(methylene)]-bis-1,4,8,11-tetraazacyclotetra-decane(Johnson Matthey); KNI-272: (2S,3S)-3-amino-2-hydroxy-4-phenylbutyric acid-containing tripeptide; L-697,593 ;5-ethyl-6-methyl-3-(2-phthalimido-ethyl)pyridin-2(1H)-one; L-735,524:hydroxy-amino-pentane amide HIV-1 protease inhibitor (Merck); L-697,661; 3-{[(-4,7-dichloro-1,3-benzoxazol-2-yl)methyl]amino}-5-ethyl-6-methyl-pyridin-2(1 H)-one; L-FDDC: (−)-β-L-5-fluoro-2′,3 ′-dideoxycytidine; L-FDOC:(−)-β-L-5-fluoro-dioxolane cytosine; MKC442:6-benzyl-1-ethoxymethyl-5-isopropyluracil (I-EBU; Triangle/Mitsubishi); Nevirapine:11-cyclo-propyl-5,11-dihydro-4-methyl-6H-dipyridol-[3,2-b:2′,3′-e]-diazepin-6-one (Boehringer-Ingelheim); NSC648400: 1-benzyloxymethyl-5-ethyl-6-(alpha-pyridylthio)uracil (E-BPTU); P9941: [2-pyridylacetyl-IlePheAla-y(CHOH)]2 (Dupont Merck); PFA: phosphonoformate (foscamet; Astra); PMEA: 9-(2-phosphonylmethoxyethyl)adenine (Gilead); PMPA: (R)-9-(2-phosphonylmethoxypropyl)adenine (Gilead); Ro 31-8959: hydroxyethylamine derivative HIV-1 protease inhibitor (Roche); RPI-312: peptidyl protease inhibitor, 1-[(3s)-3-(n-alpha-benzyloxycarbonyl)-1-asparginyl)-amino-2-hydroxy-4-phenylbutyryl]-n-tert-butyl-l-proline amide; 2720: 6-chloro-3,3-dimethyl-4-(isopropenyloxycarbonyl)-3,4-dihydro-quinoxalin-2-(1H)-thione; SC-52151: hydroxy-ethylurea isostere protease inhibitor (Searle); SC-55389A: hydroxyethyl-urea isostere protease inhibitor (Searle); TIBO R82150: (+)-(5S)-4,5,6,7-tetrahydro-5-methyl-6-(3-methyl-2-butenyl)-imidazo-[4,5,1-jk]-[1,4]-benzodiazepin-2(1H)-thione (Janssen); TIBO 82913: (+)-(5S)-4,5,6,7,-tetrahydro-9-chloro-5-methyl-6-(3-methyl-2-butenyl)imidazo[4,5,1jk]-[1,4]benzo-diazepin-2(1H)-thione (Janssen); TSAO-m3T:[2′,5′-bis-O-(tert-butyldimethylsilyl)-3′-spiro-5′-(4′-amino-1′,2′-oxathiole-2′,2′-dioxide)]-ÿ-D-pento-furanosyl-N3-methyl-thymine;U90152:1-[3-[(1-methylethyl)-amino]-2-pyridinyl]-4-[[5-[(methylsulphonyl)-amino]-1H-indol-2yl]carbonyl]piperazine;UC:thiocarboxanilide derivatives (Uniroyal); UC-781: N-[4-chloro-3-(3-methyl-2-butenyloxy)phenyl]-2-methyl-3-furan-carbothio-amide UC-82: N-[4-chloro-3-(3-methyl-2-butenyloxy)phenyl]-2-methyl-3-thiophene-carbothioamide; VB 11,328: hydroxyethyl-sulphonamide protease inhibitor (Vertex); VX-478:hydroxyethylsulphonamide protease inhibitor (Vertex); XM 323: cyclic urea protease inhibitor (Dupont Merck).

Other therapeutic agents for use in combination or alternation with the plasma or serum fraction of the present invention include: 204937 (NRTI)(GlaxoSmithKline); 640385 (VX-385) (aspartyl protease inhibitor) (GlaxoSmithKline); 695634/678248 (NNRTI) (GlaxoSmithLine); GSK-873,140 (ONO-4128) (CCR5 antagonist)(GlaxoSmithLine); AG-1859 (Pfizer); Alferon LDO® (Hemispherz BioPharma); AMD070 (Anormed); Beta-L-Fd4C (Achillion Pharmaceuticals); calanolide A (NNRTI)(Sarawak MediChem); capravirine (NNRTI)(Pfizer); Doxovir™ (Redox Pharmacueticals); elvucitabine (NRTIs) (Achillon Pharmaceuticals); entecavir (BMS 200475)(Bristol Myers Squibb); IFN-alpha lozenge (natural human interferon-alpha) (Amarillo Biosciences); Kaletra® lopinavir/ritonavir (new dosing regimen)(Abbott Laboratories); KP-1461 (Koronis Pharmaceuticals); ME-609 (Medivir); MIV-310 (Medivir); PA-457 (Panacos Pharmaceuticals); PCL-016 (Novactyl); PRO 140 (Progenics Pharmaceuticals); PRO 542 (Progenics Pharmaceuticals); PRO 2000 (Indevus Pharmaceuticals); Proleukin (Interleukin-2/Proleukin®/IL-2)(Chiron Corporation); QR-437 (Quigley Pharma); Racivir® (Pharmasset); Reverset™ (Incyte); SCH-D (Schering-Plough); SPD754 (Shire Pharmaceuticals); SPD756 (Shire Pharmaceuticals); SPO1A (Samaritan Pharmaceuticals); tipranavir (PI) (Boehringer Ingelheim); TMC114 (PI) (Tibotec); TMC125 (Tibotec); T-1249; UK-427,857 (Pfizer); Valtrex™ XR valacyclovir (modified release) (GlaxoSmithKIine); CCR5 co-receptor/entry inhibitors; CXCR4 co-receptor/entry inhibitors; and integrase inhibitors.

(ii) Anti-HBV Agents

In one embodiment, the plasma or serum fraction of the present invention can be administered alone or can be administered in combination or alternation with another anti-HBV agent or agent used to treat an HBV-related condition, opportunistic infection or co-infection.

The plasma or serum fraction of the present invention can be administered in combination or alternation with any anti-HBV agent or agent used to treat a related condition. FDA-approved HBV therapies include: Intron A (interferon α-2b, Schering-Plough); HepSera (adefovir dipivoxil, Gilead Sciences); Epivir-HBV (lamivudine, 3TC, GlaxoSmithKline); AdefoBaraclude (entercavir, Bristol-Myers Squibb) and Pegasys (peginterferon alfa-2a, Roche Labs).

Other anti-HBV agents suitable for use in combination or alternation with the plasma or serum fraction of the present invention include but are not limited to: Tenofovir (Gilead Sciences); Amdoxivir (DAPD Gilead Sciences); Racivir (Pharmasset); Valine LDC (val-LdC, Idenix Pharmaceuticals), ACH-126,443 (Elvucitabine, beta-LFd4C, Achillion Pharmaceuticals), LB80380; LG80314; Actimune; Pentacept (Pharmasset); Coviracil (emtricitabine; FTC)(Gilead Sciences; Entecavir (Bristol-Myers Squibb); Clevudine (L-FMAU) (Gilead Sciences); BAM 205 (Novelos); AM 365 (Amrad); DADP (Gilead Sciences); emtricitabine (FTC, Gilead); telbivudine (Ldt, Idenix); clevudine (L-FMAU, Gilead Sciences); LdT (telbavudine, Idenix); XTL 001 (XTL Biopharm); Theradigm (Eppimune); Zadaxin (thymosin)(SciClone); EHT 899 (Enzo Biochem); HBV DNA Vaccine (Powderject); MCC 478 (Eli Lilly); valLdC (valtorcitabine, Idenix); MIV 210 (Medivir); ICN 2001 (INC); flouroL and D nucleosides (Pharmasset); Racivir (Pharmasset); Robustaflavone (Advanced Life Sciences); HepX-B (XTL Biopharma); HE200 (Hollis Eden); Heapavir B (RibaPharm); HB DNA Vaccine (Powderject); Pradefovir (Valeant);

The plasma or serum fraction of the present invention is also suitable for us in combination or alternation with post exposure prophylaxis and post-liver transplant agents, including but not limited to BayHepB (Bayer); Nabi-HB (Nabi), as well as preventive vaccines for HBV including but not limited to Recombivax HB (Merck); Energix-B (GlaxoSmithKline); Twinrix (GlaxoSmithKline); Pediarix (GlaxoSmith Kline).

The serum or plasma fraction of the present invention can be used in combination or alternation with immunomodulatory agents. Immunomodulatory agents include, but are not limited to, a molecule such as a chemokine or cytokine that affects either directly or indirectly an immune response. Non-limiting examples of immunomodulators include TH1 cytokines, and in particular, interferon, interferon-α, purified interferon-α, interferon-α2a, interferon-α2b, interferon-β, interferon-γ, consensus interferon, pegylated interferon, pegylated interferon-α, granulocyte macrophage colony-stimulating factor, interleukin, interleukin-2, and interleukin-12. In one embodiment, the immunomodulator is interferon, e.g., interferon-γ.

The serum or plasma fraction of the present invention can be used to treat HBV+ patients is co-infected or triple infected with another virus. Non-limiting examples of HBV co-infections that can be treated with the plasma or serum fraction of the present invention include HBV/HIV, HBV/HCV, HBV/HWV/HCV. For the treatment of such co-infections, the plasma or serum fraction can be administered in combination or alternation with agents shown to be effective against the co-infected or triple infected viruses, including agents effective against HBV and HIV.

(iii) Anti-HCV Agents

In another embodiment, the plasma or serum fraction of the present invention can be administered alone or in combination with an anti-HCV agent or agent that is used to treat a related condition or concomitant infection for treatment or prevention of HCV infection or related conditions. FDA approved monotherapy and combination therapy for the treatment of HBV includes Intron A (interferon alfa 2-b); Roferon (interferon alfa-2a); Infergen (consensus interferon); Pegasys (pegylated interferon alfa-2b); PEG-Intron (Peginterferon alfa-2b); Pegasys+Copegus (peginterferon alfa-2a+ribavirin); PEG-Intron+Rebetol (peginterferon alfa-2b+ribavirin); Intron A+Rebetol (standard interferon alpha-2b+ribavirin); Roferon A+Ribavirin (interferon alfa-2a=ribavirin); and Rebetol (rivarin)/Copegus (rivarin)

Other agents suitable for use in combination with the plasma or serum fraction of the present invention for treatment of HCV infection and related conditions include, without limitation: (1) an interferon and/or ribavirin; (2) substrate-based NS3 protease inhibitors; (3) non-substrate-based inhibitors; (4) thiazolidine derivatives; (5) phenanthrenequinone; (6) selective NS3 inhibitors; (7) pestivirus, flavivirus and HCV helicase inhibitors; (8) pestivirus, flavivirus and HCV polymerase inhibitors; (9) antisense phosphorothioate oligodeoxynucleotides; (10) inhibitors of IRES-dependent translation; (11) nuclease-resistant ribozymes; and (12) other miscellaneous compounds including 1-amino-alkylcyclohexanes (U.S. Pat. No. 6,034,134 to Gold et al.), alkyl lipids (U.S. Pat. No. 5,922,757 to Chojkier et al.), vitamin E and other antioxidants (U.S. Pat. No. 5,922,757 to Chojkier et al.), squalene, amantadine, bile acids (U.S. Pat. No. 5,846,964 to Ozeki et al.), N-(phosphonoacetyl)-L-aspartic acid, (U.S. Pat. No. 5,830,905 to Diana et al.), benzenedicarboxamides (U.S. Pat. No. 5,633,388 to Diana et al.), polyadenylic acid derivatives (U.S. Pat. No. 5,496,546 to Wang et al.), 2′,3′-dideoxyinosine (U.S. Pat. No. 5,026,687 to Yarchoan et al.), and benzimidazoles (U.S. Pat. No. 5,891,874 to Colacino et al.); and (13) PEGASYS® (pegylated interferon alfa-2a) by Roche, INFERGEN® (interferon alfacon-1) by InterMune, OMNIFERON® (natural interferon) by Viragen, ALUFERON® by Human Genome Sciences, REIF® (interferon beta-1a) by Ares-Serono, Omega Interferon by BioMedicine, Oral Interferon Alpha by Amarillo Biosciences, Interferon gamma-b1 by InterMune, Interleukin-10 by Schering-Plough, IP-501 by Interneuron, Merimeodi VX-497 by Vertex, AMANTADINE® (Symmetrel) by Endo Las Solvay, HEPTAZYME® by RPI, IDN-6556 by Idun Pharma., XTL-002 by XTL., HCV/MF59 by Chiron, CIVACIR®& by NAI, LEVOVIRIN® by ICN, VIRAMIDINE® by ICN, ZADAXIN® (thymosin alfa-1) by Sci Clone, CEPLENE® (histamine dihydrochloride) by Maxim, VX 950/LY 570310 by Vertex/Eli Lilly, ISIS 14803 by Isis Pharmaceutical/Elan, IDN-6556 by Idun Pharmaceuticals, Inc. and JTK 003 by AKROS Pharma.

Non-limiting examples of the classes of drugs referenced above include:

Inteferons: Albuferon (Human Genome Sciences); Multiferon (Viragen); Peg-alfacon (InterMune); Omega interferon (Biomedicines); Medusa Interferon (Flamel Technologies); Rebif (Ares Serono); oral interferon alfa(Amarillo Biosciences)

Ribavin Alternatives: viramidine (Valeant Pharmaceuticals); merimepodib [VX-497] (Vertex Pharmaceuticals); mycophenolic acid (Roche Pharmaceuticals); amantadine (Endo Labs, etc)

Substrate-based NS3 protease inhibitors: (Attwood et al., Antiviral peptide derivatives, PCT WO 98/22496, 1998; Attwood et al., Antiviral Chemistry and Chemotherapy 1999, 10, 259-273; Attwood et al., Preparation and use of amino acid derivatives as anti-viral agents, German Patent Pub. DE 19914474; Tung et al. Inhibitors of serine proteases, particularly hepatitis C virus NS3 protease, PCT WO 98/17679), including alphaketoamides and hydrazinoureas, and inhibitors that terminate in an electrophile such as a boronic acid or phosphonate (Llinas-Brunet et al, Hepatitis C inhibitor peptide analogues, PCT WO 99/07734) are being investigated. Non-substrate-based NS3 protease inhibitors such as 2,4,6-trihydroxy-3-nitro-benzamide derivatives (Sudo K. et al., Biochemical and Biophysical Research Communications, 1997, 238, 643-647; Sudo K. et al. Antiviral Chemistry and Chemotherapy, 1998, 9, 186), including RD3-4082 and RD3-4078, the former substituted on the amide with a 14 carbon chain and the latter processing apara-phenoxyphenyl group are also being investigated.

Sch 68631, a phenanthrenequinone, is an HCV protease inhibitor (Chu M. et al., Tetrahedron Letters 37:7229-7232, 1996). In another example by the same authors, Sch 351633, isolated from the fungus Penicillium griscofuluum, was identified as a protease inhibitor (Chu M. et al., Bioorganic and Medicinal Chemistry Letters 9:1949-1952). Nanomolar potency against the HCV NS3 protease enzyme has been achieved by the design of selective inhibitors based on the macromolecule eglin c. Eglin c, isolated from leech, is a potent inhibitor of several serine proteases such as S. griseus proteases A and B, α-chymotrypsin, chymase and subtilisin. Qasim M. A. et al., Biochemistry 36:1598-1607, 1997.

Several U.S. patents disclose protease inhibitors for the treatment of HCV. For example, U.S. Pat. No. 6,004,933 to Spruce et al. discloses a class of cysteine protease inhibitors for inhibiting HCV endopeptidase. U.S. Pat. No. 5,990,276 to Zhang et al. discloses synthetic inhibitors of hepatitis C virus NS3 protease. The inhibitor is a subsequence of a substrate of the NS3 protease or a substrate of the NS4A cofactor. The use of restriction enzymes to treat HCV is disclosed in U.S. Pat. No. 5,538,865 to Reyes et al. Peptides as NS3 serine protease inhibitors of HCV are disclosed in WO 02/008251 to Corvas International, Inc, and WO 02/08187 and WO 02/008256 to Schering Corporation. HCV inhibitor tripeptides are disclosed in U.S. Pat. Nos. 6,534,523, 6,410,531, and 6,420,380 to Boehringer Ingelheim and WO 02/060926 to Bristol Myers Squibb. Diaryl peptides as NS3 serine protease inhibitors of HCV are disclosed in WO 02/48172 to Schering Corporation. Imidazoleidinones as NS3 serine protease inhibitors of HCV are disclosed in WO 02/08198 to Schering Corporation and WO 02/48157 to Bristol Myers Squibb. WO 98/17679 to Vertex Pharmaceuticals and WO 02/48116 to Bristol Myers Squibb also disclose HCV protease inhibitors.

Thiazolidine derivatives which show relevant inhibition in a reverse-phase HPLC assay with an NS3/4A fusion protein and NS5A/5B substrate (Sudo K. et al., Antiviral Research, 1996, 32, 9-18), especially compound RD-1-6250, possessing a fused cinnamoyl moiety substituted with a long alkyl chain, RD4 6205 and RD4 6193. Thiazolidines and benzanilides identified in Kakiuchi N. et al. J. EBS Letters 421, 217-220; Takeshita N. et al. Analytical Biochemistry, 1997, 247, 242-246.

A phenanthrenequinone possessing activity against protease in a SDS-PAGE and autoradiography assay isolated from the fermentation culture broth of Streptomyces sp., Sch 68631 (Chu M. et al., Tetrahedron Letters, 1996, 37, 7229-7232), and Sch 351633, isolated from the fungus Penicillium griscofuluum, which demonstrates activity in a scintillation proximity assay (Chu M. et al., Bioorganic and Medicinal Chemistry Letters 9, 1949-1952).

Helicase inhibitors (Diana G. D. et al., Compounds, compositions and methods for treatment of hepatitis C, U.S. Pat. No. 5,633,358; Diana G. D. et al., Piperidine derivatives, pharmaceutical compositions thereof and their use in the treatment of hepatitis C, PCT WO 97/36554).

Nucleotide polymerase inhibitors and gliotoxin (Ferrari R. et al. Journal of Virology, 1999, 73, 1649-1654), and the natural product cerulenin (Lohmann V. et al., Virology, 1998, 249, 108-118).

Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the HCV RNA (Alt M. et al., Archives of Virology, 1997, 142, 589-599; Galderisi U. et al., Journal of Cellular Physiology, 1999, 181, 251-257);

Inhibitors of IRES-dependent translation (Ikeda N et al., Agent for the prevention and treatment of hepatitis C, Japanese Patent Pub. JP-08268890; Kai Y. et al. Prevention and treatment of viral diseases, Japanese Patent Pub. JP-10101591);

Ribozymes, such as nuclease-resistant ribozymes (Maccjak, D. J. et al., Hepatology 1999, 30, abstract 995) and those disclosed in U.S. Pat. No. 6,043,077 to Barber et al., and U.S. Pat. Nos. 5,869,253 and 5,610,054 to Draper et al.

Immunomodulatory agents including, e.g., TH1 cytokines, interferon, interferon-α, purified interferon-α, interferon-α2a, interferon-α2b, interferon-β, interferon-γ, consensus interferon, pegylated interferon, pegylated interferon-α, granulocyte macrophage colony-stimulating factor, interleukin, interleukin-2, interleukin-12, thymosine alpha-1 (SciClone); histamine dihydrochloride (Maxim Pharmaceuticals); E1 vaccine (Innogenetics); IC-41 (Intercell); HCV-MF59 (Chiron).

Other anti-HCV agents suitable for use in combination or alternation with the plasma or serum fraction of the present invention for treatment of HCV include, but are not limited to: actimmune; Actilon (Coley); albuferon; ANA-971 (Anadys); ANA-245 (Anadys); artificial liver support system (ALSS); BILN-2061 (Boehringer Ingelheim); colchine; Enbrel (Wyeth Ayerst); HCV 896 (ViriPharma); high dose standard interferon; IDN 6556 (caspase inhibitor); interferon beta; interleukin 10 (IL-10) and (IL-11)); IP-501 (Indevus Pharmaceuticals); ID-6556 (Idun Pharmaceuticals); ISIS 14803 (ISIS Pharmaceuticals); JDK-003 (Akros Pharmaceuticals); milk thistle; NM-283 (Valopicitabine)(Idenix Pharmaceuticals); nucleoside HCV polymerase inhibitors; pirfenidone (PFD); Rituximab (Genetech); SCH 6 (HCV protease inhibitor)(Schering Plough); Tarvacin (Pergrine); viscum album; VX-950 (Vertex Pharmaceuticals); XLT-6865 (XTL); Actimmune+Pegasys or Infergen; Amantadine+standard Interferon±ribavirin; Amantadine+Pegylated interferon±ribavirin; Celgosivir+PegIntron; CellCept (mycophenotate mofetil)+Pegasys; Daily Infergen+ribavirin in nonresponders; Darbepoetin alfa+ribavirin; Dose escalation of standard Interferon+ribavirin; Dose escalaton of Pegylated IFN+ribavirin; Low dose of Pegylated Interferon+ribavirin+erythropoietin; Infergen+ribavirin; Interferon Alfa-2b+Ribavirin with Lactoferrin; Interferon alfa+ketoprofen; Interferon alfa and 200 mg daily ribavirin; Intron A+cytokines; Interleukin 2 (IL-2)+Peg-Intron+ribavirin; Levovirin+Pegasys; Lactoferrin+standard Interferon+ribavirin; Merimepodib+standard Interferon or peginterferon+ribavirin; Nucleoside HCV polymerase inhibitors; Pegylated interferon+viramidine; Pegylated Form of Consensus IFN (PEG-alfacon); Procrit (epoetin alfa) for treatment-related anemia; Ribaspheret (ribavirin)+IFN; Ribozymes; Sequential treatment with ribavirin and Interferon Alfa; Viramidine+IFN; Vitamin E and C+IFN+ribavirin; Zadaxin (thymalfasin).

(iv) Anti-Respiratory Virus Agents

In another embodiment, the plasma or serum fraction of the present invention can be administered alone or in combination with an agent that treats or prevents a respiratory virus or an agent that treats or prevents a related condition or co-infection. Non-limiting examples of suitable agents include:

Anti-influenza agents: any agents used to treat or prevent influenza, including, but not limited to (i) M2 proton channel inhibitors including amantadine (Symmetrel, Lysovir) and rimantadine (Flumavine); (ii) neuraminidase inhibitors including zanamivir (Relenza), oseltamivir (Tamiflu, GS4104), sialic acid analogues (GS4071), RWJ-270201(BCX-1812), A-315675, GG167, neuraminin; influenza vaccine including Fluzone (Aventis Pasteur, Inc.) and Fluvirin (Evans Vaccines) as well as the nasal influenza vaccine FluMist (Medlmmune)

Anti-RSV agents: any agents used to treat or prevent respiratory syncytial virus (RSV) including, for example, Ribavirin (Virazole®); RSV-IG (Respigam®; MedImmune, Inc.); palivizumab (Synagis®; MedImmune, Inc.); the triphenolVP-14637 (ViroPharma); the benzimidazole derivative R-170591 (Janssen Pharmaceutical); the disulfonated stilbenes CL387626 and RFI-641 (Wyeth-Ayerst); and RSV vaccine

Anti-parainfluenza agents: any agents used to treat or prevent infections with a parainfluzena virus including, e.g., ribavirin; parainfluenza viral vaccines, including inactivated viral vaccines and intransal vaccines.

Anti-rhinovirus agents: any agent used to treat or prevent infections with rhinovirus, including e.g., Pleconaril (Picovir), a novel viral capsid-binding inhibitor; protease inhibitors including 2S protease inhibitors (e.g., ) and 3C protease inhibitor (e.g., AG7088 by Agouron Pharmaceuticals); interferon alpha; Pirodavir, substituted phenoxy-pyridazinamine; WIN 54954, a methylisoxazole derivative; soluble ICAM-1; virus receptor blockers; antireceptor antibody; rhinovirus vaccine

Anti-adenovirus agents: any agents used to treat or prevent infections with an adenovirus, e.g., ribavirin and adenovirus vaccines

Anti-coronavirus agents: any agent used to treat or prevent infections with a coronavirus, including protease inhibitors (e.g., AG7088); steroids (e.g., corticosteroid therapy); Ribavirin, alone or in combination with gucocorticoids or methylprednisolone; interferon; bovine surfactant therapy (Moller J C et al. Intensive Care Med. 2003 29: 437-436); passive immunization with anti-coronavirus neutralizing antibodies, including SARco-V neutralizing antibodies; coronovirus vaccination, including SARco-V vaccination.

The plasma or serum fraction of the present invention can also be used alone or in combination or alternation with supportive therapy for respiratory viral infections, including for example, symptomatic treatment with analgesics (e.g., Ibuprofen, Acetaminophen) decongestants (e.g. Pseudoephedrine, Phenylephrine), antihistamines (e.g., Brompheniramine), bronchiodialators (e.g., metaproterenol, albuterol), oxygen therapy, vitamin (e.g., Vitamin A, Vitamin C), and antitussives (e.g., Dextromethorphan, Codeine).

(iv) Anti-Herpes Agents

In another embodiment, the plasma or serum fraction of the present invention can be administered alone or in combination with an anti-Herpesvirdiae agent or agent that is used to treat a related condition or concomitant infection. Suitable anti-Herpesvirdiae agents include, without limitation, anti-HSV-1 agents, anti-HSV-2 agents, anti-CMV agents, anti-EVC agents and anti-VZV agents.

Suitable agents include, without limitation, transcriptase inhibitors, IMPDH (inosine monophosphate dehydrogenase) inhibitors, inhibitors of virus adsorption entry, inhibitors of fusion with host cells, anti-sense oligonucleotides, immunoglobins, and immunostimulatory agents.

Other non-limiting examples of antiviral agents that can be used in combination and/or alternation with the compositions of the present invention include Acyclovir (ZOVIRAX™), Famciclovir (FAMIVR™), Valacyclovir (VALTREX™), edoxudine (VIROSTA™), ganciclovir, foscamet, cidovir (vistide), Vitrasert, Formivirsen, HPMPA (9-(3-hydroxy-2-phosphonomethoxypropyl)adenine), PMEA (9-(2-phosphonomethoxyethyl)adenine), HPMPG (9-(3-Hydroxy-2-(Phosphonomethoxy)-propyl)guanine), PMEG (9-[2-(phosphonomethoxy)ethyl]-guanine), HPMPC (1-(2-phosphonomethoxy-3-hydroxypropyl)-cytosine), ribavirin, EICAR (5-ethynyl-1-beta-D-ribofuranosylimidazole-4-carboxamide), pyrazoftrin (3-[beta-D-riboftiranosyl]-4-hydroxy-pyrazole-5-carboxamide), 3-Deazaguanine GR-92938X (1-β-D-ribofuranosyl-pyrazole-3,4-dicarboxamide), LY253963 (1,3,4-thiadiazol-2-yl-cyanamide), RD3-0028 (1,4-dihydro-2,3-Benzodithiin), CL387626 (4,4′-bis[4,6-di[3-aminophenyl-N,N-bis(2-carbamoylethyl)-sulfonilimino]-1,3,5-triazin-2-ylamino-biphenyl-2,2′-disulfonic acid dissodium salt), BABIM (Bis[5-Amidino-2-benzimidazolyl]methane), and NIH351.

Non-limiting examples of anti-HSV agents that can be used in combination and/or alternation with the compositions of the present invention include Acyclovir (ZOVIRAX™), Famciclovir (FAMIVR™), Valacyclovir (VALTREX™), edoxudine (VIROSTA™).

Non-limiting examples of anti-VZV agents that can be used in combination and/or alternation with the compositions of the present invention include Acyclovir (ZOVIRAX™), Famciclovir (FAMIVR™), Valacyclovir (VALTREX™), edoxudine (VIROSTA™).

Non-limiting examples of anti-CMV agents that can be used in combination and/or alternation with the compositions of the present invention include acyclovir, ganciclovir, foscamet, cidovir (vistide), Vitrasert, Formivirsen, HPMPA, PMEA, HPMPG, PMEG, and HPMPC.

Non-limiting examples of anti-EBV agents that can be used in combination and/or alternation with the plasma or serum fraction of the present invention include acyclovir (ACV), AZT, 3TC, d4T, ganciclovir (GCV or DHPG) and its prodrugs (e.g., valyl-ganciclovir), E-5-(2-bromovinyl)-2 ′-deoxyuridine (BVDU), (E)-5-vinyl-1-β-D-arabonosyluracil (VaraU), (E)-5-(2-bromovinyl)-1-β-D-arabinosyluracil (BV-araU), 1-(2-deoxy-2-fluoro-β-D-arabinosyl)-5-iodocytosine (D-FIAC), 1-(2-deoxy-2-fluoro-β-L-arabinosyl)-5-methyluracil (L-FMAU), (S)-9-(3-hydroxy-2-phosphonylmethoxy-propyl)adenine [(S)-HPMPA], (S)-9-(3-hydroxy-2-phosphonylmethoxypropyl)-2,6-diaminopurine [(S)-HPMPDAP], (S)-1-(3-hydroxy-2-phosphonyl-methoxypropyl)-cytosine [(S)-HPMPC, or cidofivir], and (2 S,4S)-1-[2-(hydroxymethyl)-1,3-dioxolan-4-yl]-5-iodouracil (L-5-IoddU).

In one embodiment, the plasma or serum fraction of the present invention may be employed together with at least one polymerase inhibitor.

In another embodiment, the plasma or serum fraction of the present invention can be used in combination or alternation with an anti-proliferative agent for the treatment of a herpes virus infection or related condition. The anti-proliferative agent, as used herein, is any agent that decreases the hyperproliferation of cells. Any of the anti-proliferative agents listed below, or any other such agent known or discovered to exhibit an anti-proliferative effect can be used in accordance with this invention.

Proliferative disorders are currently treated by a variety of classes of compounds including alkylating agents, antimetabolites, natural products, enzymes, biological response modifiers, miscellaneous agents, hormones and antagonists, such as those listed below. Examples, include, but are not limited to:

Monoclonal Antibodies: monoclonal antibodies directed to proliferating cells such as Rituximab (anti-CD20) for B-cell tumors.

Alkylating Agents: nitrogen mustards such as mechlorethamine (Hodgkin's disease, non-Hodgkin's lymphomas), Cyclophosphamide Ifosfamide (acute and chronic lymphocytic leukemias, Hodgkin's disease, non-Hodgkin's lymphomas, multiple myeloma, neuroblastoma, breast, ovary, lung, Wilms' tumor, cervix, testis, soft-tissue sarcomas), melphalan (L-sarcolysin) (multiple myeloma, breast, ovary), chlorambucil (chronic lymphocytic leukemia, primary macroglobulinemia, Hodgkin's disease, non-Hodgkin's lymphomas). Ethylenimines and methylmelamines such as hexamethylmelamine (ovary), Thiotepa (bladder, breast, ovary). Alkyl sulfonates such as busulfan (chronic granuloytic leukemia). Nitrosoureas such as Cannustine (BCNU) (Hodgkin's disease, non-Hodgkins lymphomas, primary brain tumors, multiple myeloma, malignant melanoma), Lomustine (CCNU) (Hodgkins disease, non-Hodgkins lymphomas, primary brain tumors, small-cell lung), Semustine (methyl-CCNU) (primary brain tumors, stomach, colon), Streptozocin (streptozocin) (malignant pancreatic insulinoma, malignant carcinoin). Triazenes such as Dacarbazine (DTIC; dimethyltriazenoimid-azolecarboxamide) (malignant melanoma, Hodgkins disease, soft-tissue sarcomas).

Anti-metabolites: Folic acid analogs such as methotrexate (amethopterin) (acute lymphocytic leukemia, choriocarcinoma, mycosis fungoides, breast, head and neck, lung, osteogenic sarcoma). Pyrimidine analogs such as fluorouracil (5-fluorouracil; 5-FU) Floxuridine (fluorodeoxyuridine; FUdR) (breast, colon, stomach, pancreas, ovary, head and neck, urinary bladder, premalignant skin lesions) (topical), Cytarabine (cytosine arabinoside) (acute granulocytic and acute lymphocytic leukemias). Purine analogs and related inhibitors such as mercaptopurine (6-mercaptopurine; 6-MP) (acute lymphocytic, acute granulocytic and chronic granulocytic leukemia), thioguanine (6-thioguanine: TG) (acute granulocytic, acute lymphocytic and chronic granulocytic leukemia), Pentostatin (2′-deoxycyoformycin) (hairy cell leukemia, mycosis fungoides, chronic lymphocytic leukemia). Vinca alkaloids such as Vinblastine (VLB) (Hodgkin's disease, non-Hodgkins lymphomas, breast, testis), vincristine (acute lymphocytic leukemia, neuroblastoma, Wilms' tumor, rhabdomyosarcoma, Hodgkin's disease, non-Hodgkins lymphomas, small-cell lung). Epipodophyl-lotoxins such as etoposide (testis, small-cell lung and other lung, breast, Hodgkin's disease, non-Hodgkins lymphomas, acute granulocytic leukemia, Kaposi's sarcoma), teniposide (testis, small-cell lung and other lung, breast, Hodgkin's disease, non-Hodgkins lymphomas, acute granulocytic leukemia, Kaposi's sarcoma).

Natural Products: Antibiotics such as dactinomycin (actinonmycin D) (choriocarcinoma, Wilms' tumor rhabdomyosarcoma, testis, Kaposi's sarcoma), daunorubicin (daunomycin; rubidomycin) (acute granulocytic and acute lymphocytic leukemias), doxorubicin (soft tissue, osteogenic, and other sarcomas; Hodgkins disease, non-Hodgkins lymphomas, acute leukemias, breast, genitourinary thyroid, lung, stomach, neuroblastoma), bleomycin (testis, head and neck, skin and esophagus lung, and genitourinary tract, Hodgkins disease, non-Hodgkins lymphomas), plicamycin (mithramycin) (testis, malignant hypercalcema), mitomycin (mitomycin C) (stomach, cervix, colon, breast, pancreas, bladder, head and neck). Enzymes such as L-asparaginase (acute lymphocytic leukemia). Biological response modifiers such as interferon-alfa (hairy cell leukemia, Kaposi's sarcoma, melanoma, carcinoid, renal cell, ovary, bladder, non Hodgkin's lymphomas, mycosis fungoides, multiple myeloma, chronic granulocytic leukemia), interferon-gamma, IL-2 and IL-12.

Hormones and Antagonists: including but not limited to estrogens such as diethylstibestrol ethinyl estradiol (breast, prostate); antiestrogens such as tamoxifen (breast); androgens such as testosterone propionate Fluxomyesterone (breast); antiandrogens such as flutamide (prostate); gonadotropin-releasing hormone analogs such as leuprolide (prostate).

Miscellaneous Agents: Platinum coordination complexes such as cisplatin (cis-DDP) carboplatin (testis, ovary, bladder, head and neck, lung, thyroid, cervix, endometrium, neuroblastoma, osteogenic sarcoma). Anthracenediones such as mixtozantrone (acute granulocytic leukemia, breast). Substituted ureas such as hydroxyurea (chronic granulocytic leukemia, polycythemia vera, essential thrombocytosis, malignant melanoma). Methylhydrazine derivatives such as procarbazine (N-methylhydrazine, MIH) (Hodgkins disease). Adrenocortical suppressants such as miotane (o,p′-DDD) (adrenal cortex), aminoglutethimide (breast). Adrenorticosteriods such as prednisone (acute and chronic lymphocytic leukemias, non-Hodgkins lymphomas, Hodgkin's disease, breast). Progestins such as hydroxprogesterone caproate, medroxyprogersterone acetate, megestrol acetate (endometrium, breast). Demethylating agents such as azacytidine. PKC activators such as bryostatins. Differentiating agents such as butyrates, retinoic acid and related retinoids. Microtubule inhibitors such as taxols and taxanes. Topoisomerase inhibitors such as topotecan. Miscellaneous compounds such as valproic Acid, HMBA, NF-kappaB Inhibitors. In another embodiment, the further antiviral agent is an immunostimmulatory agent.

C. Adjuvants that Can Be Used in Combination and/or Alternation With the Composition of the Present Invention

In addition, if desired, the composition may contain auxiliary substances such as wetting or emulsifying agents, pH buffering agents or adjuvants that enhance the effectiveness of the composition. As non-limiting examples, the plasma or serum fraction can be administered in combination or alternation with any of the following known adjuvants.

Adjumer (PCPP salt; polyphosphazene; polyidi(carboxylatophenoxy)lphosphazene) which may be administered in the soluble form as an adjuvant for parenteral formulations or in the crosslinked form as a microsphere hydrogel for mucosal formulations. It induces a sustained antibody response after a single parenteral immunization and these antibody responses include antigen specific IgG1 and IgG2a. with sustained IgG and IgA responses also induced in after mucosal immunization. Algal Glucan (also known as β-glucan or glucan) is administered with antigen for enhancement of both humoral and cell-mediated immunity. β-Glucans exert their immunostimulatory activities by binding to specific P-glucan receptors on macrophages. This ligand-receptor interaction results in macrophage activation and, in certain formulations, promotes antigen targeting. Algammulin (gamma inulin/alum composite adjuvant) is used in formulations as a primary adjuvant and stimulates immune responses by causing ligation of leukocyte-surface complement receptors (CR) via known biochemical mechanisms, thus placing the antigen close to activated leukocytes. Addition of Algarnmulin is known to enhance both humoral and cell-mediated immunity from either Th1 or Th2 pathways, depending on the weight ratio of inulin to Alhydrogel. Avridine (N,N-dioctadecyl-N′,N′-bis(2-hydroxyethyl) propanediamine; CP20,961) may be incorporated into a liposomal preparation; into aqueous suspensions from alcoholic solution; in Intralipid, an aqueous soybean oil emulsion vehicle; other vegetable and mineral oil vehicles; in Tween 80 dispersions in saline; in saline suspension with alum-precipitated antigen. It has been shown to cause humoral and cellular immunity, proliferation of B and T lymphocytes, protective immunity, activation of macrophages, induction of interferon, enhancement of mucosal immunity when administered orally/enterically with antigen, adjuvanticity with a variety of antigens, induction of IgG2a and IgG2b isotypes. BAY R1005 (N-(2-Deoxy-2-L-leucylamino-β-D-glucopyranosyl)-N-octadecyldodecanoylamide hydroacetate) can be used as a primary adjuvant. BAY R1005 in combination with purified virus vaccines or subunit vaccines led to increased protection of virus-challenged mice. The increase in antibody synthesis induced by BAY R1005 is specifically dependent on the antigen and it acts on the proliferation of B lymphocytes as a second signal which has no effect until the antigen acts as a first signal. BAY R 1005 is capable of activating B lymphocytes without the helper function of T lymphocytes. In mice parenteral immunization with recombinant urease mixed with BAY R1005 induced strong Th1 and Th2 responses and thereby elicited better protection against Helicobacter pylori infection than adjuvants which induced a prominent Th2 type response only (Guy, B., et al., 1998. Systemic immunization with urease protects mice against Helicobacter pylon infection. Vaccine 16:850-856.) Calcitriol (1α, 25-dihydroxyvitamin D3; 1,25-di(OH)₂D₃; 1,25-DHCC; 1α, 25-dihydroxycholecalciferol; 9,10-seco(5Z,7E)-5,7,10(19)-cholestatriene-1α,3β,25-triol) has been shown to promote the induction of mucosal and systemic immunity when incorporated into vaccine formulations. Calcium Phosphate Gel has been used as adjuvant in vaccine formulations against diphtheria, tetanus, pertussis and poliomyelitis. It adsorbs soluble antigens and presents them in a particulate form to the immune system and contains no components that are not natural constituents of the body and is very well tolerated. Cholera toxin B subunit (CTB, also known as CTB subunit) augments humoral responses by acting as an efficient carrier/delivery system and is completely non-toxic and has been used extensively in humans without negative side-effects. Cholera holotoxin (CT) has been shown to augment both humoral and cell-mediated immunity, including CTL responses, and thereby enhances MHC class I and II restricted responses. CT exerts immunomodulating effects on T cells, B cells as well as antigen-presenting cells (APC). Cholera toxin Al-subunit-ProteinA D-fragment fusion protein (CTA1-DD gene fusion protein) has proven equivalently potent as an adjuvant to the intact cholera holotoxin (CT) for humoral and cell-mediated immunity. CTA1-DD is targeted to B lymphocytes, both memory and naive cells and acts as a powerful systemic and mucosal adjuvant.

Block Copolymer P1205 (CRL1005) acts as both an adjuvant and stabilizer and forms microparticulate structures that can bind a variety of antigens via a combination of hydrophobic interactions and surface charge. Cytokine-containing Dehydration Rehydration Vesicles (Cytokine-containing Liposomes) induces both cellular and humoral immunity. Dimethyldioctadecylammonium bromide; dimethyldistearylammonium bromide (DDA-CAS Registry Number 3700-67-2) is known for stimulation immune responses against various antigens and especially delayed type hypersensitivity. DHEA (Dehydroepiandrosterone; 5-androsten-3β-ol-17-one; dehydroisoandrosterone; androstenolone; prasterone; transdehydroandrosterone; DHA) can be directly incorporated into vaccine formulations and will enhance antibody formation. DHEA can be administered systemically at the time of vaccination, or can be directly incorporated into the vaccine formulation. DMPC (Dimyristoyl phosphatidylcholine; sn-3-phosphatidyl choline-1,2-dimyristoyl; 1, 2-dimyristoyl-sn-3-phosphatidyl choline; (CAS Registry Number 18194-24-6)) and DMPG (Dimyristoyl phosphatidylglycerol; sn-3-phosphatidyl glycerol-1,2-dimyristoyl, sodium salt (CAS Registry Number 67232-80-8); 1,2-dimyristoyl-sn-3-phosphatidyl glycerol) are used in the manufacture of pharmaceutical grade liposomes, typically in combination with DMPG and/or cholesterol and are also used in adjuvant systems for vaccine formulations. DOC/Alum Complex (Deoxycholic Acid Sodium Salt; DOC/Al(OH)₃/mineral carrier complex) is a complex used as adjuvant formulation and is known to enhance the immune response to membrane proteins. Freund's Complete Adjuvant is a mixture of mineral oil (Marco 52) and emulsifier (Arlacel A [mannide monooleate]) as an emulsion of 85% mineral oil and 15% emulsifier with heat-killed antigen. Gamma Inulin is a highly specific activator of the alternative pathway of complement in vitro and in vivo included in adjuvant formulations as a primary adjuvant and also as the immune stimulant when combined as composite particles with alum in the adjuvant Algammulin. It is expected that it stimulates immune responses by causing ligation of leukocyte-surface complement receptors (CR) via known biochemical mechanisms. Addition of gamma inulin is known to enhance both humoral and cell-mediated immunity from both Th1 and Th2 pathways. Gamma inulin also has an antitumor action and an effect on natural immunity. Gerbu Adjuvant, an adjuvant based on GMDP with DDA and Zinc-L-proline have been shown to complex as synergists. GM-CSF (Granulocyte-macrophage colony stimulating factor; Sargramostim (yeast-derived rh-GM-CSF)) is a glycoprotein of 127 amino acids and recombinant human GM-CSF is produced in yeast and it differs from the natural human GM-CSF by substitution of Leu for Arg at position 23. This cytokine is a growth factor that stimulates non-nal myeloid precursors, and activates mature granulocytes and macrophages.

GMDP (N-acetylglucosaminyl-(β1-4)-N-acetylmuramyl-L-alanyl-D-isoglutamine (CAS Registry Number 70280-03-4)—Semi-synthetic. Disaccharide isolated from microbial origin, dipeptide wholly synthetic. U.S. Pat. No. 4,395,399) is known as a primary adjuvant. It has been shown to be an highly effective primary adjuvant in a range of vehicles; aqueous buffers, mineral oil, pluronic/squalane/Tween emulsions. Also effective as oral adjuvant, enhancing mucosal IgA response. Imiquimod (1-(2-methypropyl)-1H-imidazo[4,5-c]quinolin-4-amine; R-837; S26308) can be included in adjuvant formulations as a primary adjuvant component and is known to induce both humoral and cell-mediated immunity via induction of cytokines from monocytes and macrophages. ImmTher™ (N-acetylglucosaminyl-N-acetyhnuramyl-L-Ala-D-isoGlu-L-Ala-glycerol dipalmitate; DTP-GDP) is a potent macrophage activator which induces high levels of TNF, IL-1, and IL-6 both in vitro and in vivo (U.S. Pat. No. 4,950,645). Immunoliposomes prepared from Dehydration-Rehydration Vesicles (DRVs) (Immunoliposomes Containing Antibodies to Costimulatory Molecules) are composed of phosphotidylcholine/cholesterol/biotinylate d-phospotidylethanolamine (PC/CH/PEB) in a molar ration of 5:5:1. Antigen is added to the water suspension of DRV followed by repeated vortexing and lyophylization of the liposome suspension. Interferon-γ (Actimmune® (rhIFN-gamma, Genentech, Inc.); immune interferon; IFN-γ; gamma-interferon) has demonstrated higher and earlier neutralizing antibody titers, an increase in duration of neutralizing antibody titers, an increase in MHC class 11 expression on antigen presenting cells, increase in Helper T cell levels, and an improved DTH response. Interleukin-1,β(IL-10; IL-1; human Interleukin 1β mature polypeptide 117-259) is known as a primary adjuvant and is active by oral, intravenous, intraperitoneal and subcutaneous routes. It increases both T-dependent and T-independent responses to different types of antigens and can be active in both primary and secondary responses. Interleukin-2 (IL-2; T-cell growth factor; aldesleukin (des-alanyl-1, serine-125 human interleukin 2); Proleukin®; Teceleukin®) is used as a primary adjuvant, co-emulsified with antigens and lipids, with polyethylene glycol modified long acting form (PEG IL-2), or liposome encapsulated sustained release dosage form. IL-2 supports the growth and proliferation of antigen-activated T lymphocytes and plays a central role in the cascade of cellular events involved in the immune response. Proliferating T-cells also produce a variety of other lymphokines which may modulate other arms of the immune system and in view of these direct and indirect actions of IL-2 on the immune response, IL-2 functions as an adjuvant to vaccination by increasing the specific and durable response to vaccine immunogens. It may induce cellular immunity when given systemically, and IgA when administered at a mucosal surface. Interleukin-7 (IL-7) has been shown to enhance antibody production as a primary adjuvant in liposome formulated sustained release form (Bui, et al. “Effect of MTP-PE liposomes and interleukin-7 on induction of antibody and cell-mediated immune responses to a recombinant HIV-envelope protein”, J Acquired Immune Deficiency Syndrome, Aug. 7, 1994;(8):799-806.) and has also been used co-emulsified with antigen and lipids. Interleukin-12 (IL-12; natural killer cell stimulatory factor (NKSF); cytotoxic lymphocyte maturation factor (CLMF)) is used as a primary adjuvant component to enhance Th1-dependent cell-mediated immune responses including cytolytic T-lymphocyte responses.

Immune stimulating complexes (ISCOM(s)™) are a complex composed of typically Quillaja saponins, cholesterol, phospholipid, and antigen in phosphate-buffered saline (PBS). They are antigen-presenting structures that have been shown to generate long-lasting biologically functional antibody response. ISCOMs have demonstrated a protective immunity and a functional cell-mediated immune response, including Class I restricted CTLs have been reported in several systems. They have generally been administered subcutaneously or intramuscularly but non-parenteral administrations (intranasal and oral) have also proven to be effective. Liposomes (L) containing protein or Th-cell and/or B-cell peptides, or microbes with or without co-entrapped interieukin-2, BisHOP or DOTMA A, [L (Antigen)]; B, [L (IL-2 or DOTMA or BisHOP+Antigen)]; C, [L (Antigen)-mannose]; D, [L (Th-cell and B-cell epitopes)]; E, [L (microbes)] act as carrier of Th-cell peptide antigen which provides help for co-entrapped B-cell antigen to overcome genetic restriction and induce immunological memory. They may also act as carriers of attenuated or live microbial vaccines to deliver microbes and co-entrapped soluble antigens or cytokines simultaneously to antigen-presenting cells or to protect entrapped vaccines from interaction with maternal antibodies or antibodies to vaccine impurities in preimmunized subjects. Loxoribine (7-allyl-8-oxoguanosine) is known as a primary adjuvant for antibody responses to a wide variety of antigen types in a variety of species. It augments CTL-mediated, NK cell-mediated, macrophage mediated, and LAK cell-mediated cytotoxicity, induces IFN(a/b/γ, TNΦa, TNΦb, IL-1a, IL-6 and up regulates humoral immune responses in immunodeficiency. LT-OA or LT Oral Adjuvant induces both mucosal and systemic immunity (both humoral [including IgA and IgG2, isotypes] and cell-mediated) to killed microorganisms or peptide antigens mixed with it in neutral non-phosphate buffered saline, with/without sodium bicarbonate. MF59 (Squalene/water emulsion-composition: 43 mg/mL squalene, 2.5 mg/mL polyoxyethylene sorbitan monooleate (Polysorbate 80), 2.4 mg/mL sorbitan trioleate (Span 85)) in combination with a variety of subunit antigens results in elevated humoral response, increase T cell proliferation and presence of cytotoxic lymphocytes. MONTANIDE ISA 51 (Purified IFA; Incomplete Freund's adjuvant) addition induces humoral and cell-mediated immunity with various antigens. MONTANIDE ISA 720 (metabolizable oil adjuvant) induces humoral and cell-mediated immunity with various antigens. MPL™ (3-Q-desacyl-4′-monophosphoryl lipid A; 3D-MLA) is used as a primary adjuvant in adjuvant formulations. Its activity is manifested either alone in aqueous solution with antigen, or in combination with particulate vehicles (e.g., oil-in-water emulsions) and its activity may be enhanced by use of vehicle that enforces close association with antigen. MTP-PE (N-acetyl-L-alanyl-D-isoglutaminyl-L-alanine-2-(1,2-dipalmitoyl-sn-glycero-3-(hydroxy-phosphoryloxy)) ethylamide, mono sodium salt) and alternately MTP-PE liposomes are optionally a part of MF59 and are known as immunomodulators. The addition of MTP-PE to the MF59-based HIV vaccine in HIV seropositive individuals resulted in a marked increase in HIV antigen lymphocyte proliferation. Murametide (Nac-Mur-L-Ala-D-Gln-OCH3) induces granulocytosis and enhances the humoral response. Murametide displays the same profile of adjuvant activity as MDP and has been chosen for development because of its favorable therapeutic ratio. When administered in 50% water-in-oil emulsion, it mimics the activity of Freund's complete adjuvant without its side effects (U.S. Pat. No. 4,693,998.) Murapalmitine (Nac-Mur-L-Thr-D-isoGIn-sn-glyceroI dipalmitoyl) is administered in water-in-oil emulsion as an adjuvant of humoral and cell-mediated responses. D-Murapalmitine (Nac-Mur-D-Ala-D-isoGln-sn-glycerol dipalmitoyl) is a strong adjuvant of humoral and cell-mediated immunity when administered in a 50% mineral oil emulsion. NAGO is a mixture of the two enzymes-neuraminidase and galactose oxidase Ag 1:5 ratio in units of activity. It generates cell surface Schiff base-forming aldehydes on antigen presenting cells and Th-cells, thereby amplifying physiologic Schiff base formation that occurs between cell-surface ligands as an essential element in APC:T-cell inductive interaction. It is a potent non-inflammatory adjuvant with viral, bacterial and protozoal subunit vaccines, and is especially effective in the generation of cytotoxic T-cells.

Non-Ionic Surfactant Vesicles (NISV) induces both a humoral and cell-mediated immune response and preferentially stimulates the Th1 sub-population of T-helper cells. It is known to be effective with antigens within a broad size range, from short peptides to particulates, and has extremely low toxicity. Pleuran (β-glucan; glucan) has shown in experimental studies that rabbits as well as mice immunized once by coadministration of viral antigens and 60 μg of Pleuran produced at least 20-fold higher antibody titers than control animals injected with the immunogen alone (Chihara, G. et al., 1989, Lentinan as a host defense potentiator (HDP), Int. J. Immunother. 4:145-154.) PLGA, PGA, and PLA (Homo-and co-polymers of lactic and glycolic acid; Lactide/glycolide polymers; poly-lactic-co-glycolide) used in vaccine delivery have demonstrated an ability to control the release of antigen after administration, thereby eliminating or reducing the need for boost immunizations. Antigens incorporated in PLGA microspheres have exhibited enhanced and prolonged antibody activity responses compared to equivalent doses of free antigen. Pluronic L121 (poloxamer 401) enhances the presentation of antigen to cells of the immune system. PMMA (polymethyl methacrylate) is known as a primary adjuvant for all types of antigens. PODDS™ (proteinoid microspheres) serves as a vehicle for oral immunization, protecting the antigen and allowing for co-encapsulation of adjuvants with antigens in microspheres. Poly rA:Poly rU (a double helix comprised of polyadenylic acid and polyuridylic acid) is known as an adjuvant to humoral and cell-mediated immunity when given with antigen; it increases non-specific immunity to microorganisms. Polysorbate 80 may be used in emulsion vaccine formulations including MF59, SAF-1 and Antigen Formulation. Protein cochleates act as both carriers and adjuvants, providing multivalent presentation of antigens to the immune system, with maintenance of native conformation and biological activity and providing protection of antigens from degradation following oral delivery. They stimulate strong mucosal and systemic antibody, proliferative and cytotoxic responses to associated antigens. QS-21 (Stimulon™ QS-21 Adjuvant) can be used in vaccine formulations as a primary adjuvant component for enhancement of both humoral and cell-mediated immunity. Quil-A (Quil-A saponin, Quillaja saponin) is used in veterinary vaccines and for production of ISCOMs. Rehydragel HPA (High Protein Adsorbency Aluminum Hydroxide Gel; alum) and Rehydragel LV (low viscosity alluminum hydroxide gel; alum) are primary adjuvants in parenteral vaccine formulations and aluminum compounds (aluminum hydroxide, aluminum phosphate, alum) are currently the only vaccine adjuvants used in US-licensed vaccines. The use of aluminum adjuvants are accompanied by stimulation of IL-4 and stimulation of the T-helper-2 subsets in mice, with enhanced IgG1 and IgE production. S-28463 (4-Amino-otec,-dimethyl-2-ethoxymethyl-lH-imidazo[4,5-c]quinoline-1-ethanol) induces both humoral and cell-mediated immunity via induction of cytokines from monocytes and macrophages. Experimental results indicate S-28463 is about 100-fold more potent than imiquimod in antiviral models and in cytokine induction from monocytes and macrophages. Syntex Adjuvant Formulation (SAF, SAF-1, SAF-m) causes antigens to arrange on the surface of the emulsion droplets partly because of their amphipathic nature, and partly because of hydrogen bonding with poloxamer 401. The emulsion droplets also activate complement, as demonstrated by consumption of C3 and production of C3b; the latter, on the surface of droplets, targets them to antigen-presenting cells (follicular dendritic cells and interdigitating cells) in lymph nodes of the drainage chain and possibly in more distant lymphoid tissues. In this way the emulsion facilitates the presentation of antigens to responding lymphocytes (threonyl-MDP monograph.) Sclavo peptide (IL-1,β 163-171 peptide) enhances immune response to T-dependent and T-independent antigens. It is known as a primary adjuvant and may be administered i.p, i.v., s.c. or p.o and it is active either when administered separately from antigen, or admixed with antigen, or physically linked to antigen. Sendai Proteoliposomes, Sendai-containing Lipid Matrices (Sendai glycoprotein-containing vesicles; fusogenic proteoliposomes; FPLs; Sendai lipid matrix-based vaccines) are potent immunogens and have the ability to stimulate strong T helper and CD8+ cytotoxic T cell responses (CTL) to lipid bilayer-integrated glycoproteins as well as encapsulated peptides, proteins and whole formalin-fixed viruses. These vesicles also act as effective delivery vehicles for drugs and proteins.

Span 85 (Arlacel 85, sorbitan trioleate) is used as an emulsification agent in MF59 adjuvant formulation. Specol (Marcol 52 (mineral oil, paraffins, and cycloparaffins, chain length 13-22 C atoms) Span 85 (emulsifier, sorbitan trioleate) Tween 85 (emulsifier, polyoxyethylene-20-trioleate)) all are individually FDA approved for veterinary use and they function as a depot (slow release of antigen) and a polyclonal activator (independent of presence of antigen) for cells of the immune system (cytokine release). Squalane (Spinacane;Robane®;2,6,10,15,19,23-hexamethyltetracosane) is a component of Antigen Formulation (AF) and Syntex Adjuvant Formulation (SAF), and constitutes the oil component of the emulsion. Stearyl Tyrosine (octadecyl tyrosine hydrochloride) has adjuvanticity similar to aluminum hydroxide with bacterial vaccines; superior to aluminum hydroxide with viral vaccines. Theramide™ (N-acetylglucosaminyl-N-acetylinuramyl-L-Ala-D-isoGlu-L-Ala-dipalmitoxy propylamide (DTP-DPP)) is a potent macrophage activator and adjuvant. It induces IL-6, IL-12, TNF, IFN-γ, and relatively lessor quantities of IL-10. The compound preferentially induces cellular immunity. Threonyl-MDP (Termurtide™; [thr₁]-MDP; N-acetyl muramyl-L-threonyl-D-isoglutamine) induces the production of a cascade of cytokines, including IL-1a, IL-1β and IL-6. Responding lymphocytes release IL-2 and IFN-γ and the latter increases the production of antibodies of certain isotypes, including IgG2a. This isotype, and the homologous IgG1 in primates, interacts with high affinity Fcγ receptors, so that the antibodies can function efficiently in opsonizing viruses and other infectious agents for uptake by phagocytic cells. Ty Particles or Ty Virus-Like Particles present antigen in a polyvalent, particulate form. Cytotoxic T-lymphocytes are induced in the absence of any other adjuvant formulations. Walter Reed Liposomes (Liposomes containing lipid A adsorbed to aluminum hydroxide, [L(Lipid A+Antigen)+Alum]) have been shown to induce both humoral and cell-mediated immunity. Liposomes containing lipid A provide a very potent adjuvant activity. Adsorption of liposomes containing lipid A to aluminum hydroxide gel contributes additional strong adjuvant activity.

D. Pharmaceutical Compositions

Humans suffering from a viral infection including, but not limited to, an HIV infection, an HBV infection, an HCV infection, a respiratory virus infection or a herpes virus infection, can be treated by administering to the patient an effective amount of the defined plasma or serum fraction. In one embodiment of the present invention, the plasma or serum fraction is delivered alone. In another embodiment of the present invention, the plasma or serum fraction can be formulated in the presence of a pharmaceutically acceptable carrier, excipient or diluent. Examples of suitable pharmaceutical carriers are described in “Remington's Pharmaceutical Sciences” by E. W. Martin. The formulation should suit the mode of administration, according to principles well known in the art.

Treatment comprising administering the composition of the present invention subcutaneously, parenterally, intravenously, intraarterially, intraperitoneal, intracorporeal injection, intraventricular, intrathecally, intramuscularly, subcutaneously, intranasally, intravaginally, orally, topically (i.e., by powders, ointments, drops or transdermal patch) or by any other effective method of administration. In one embodiment, the treatment is administered subcutaneously (e.g., by subcutaneous injection).

The composition can be formulated for parenteral administration, which involves the administration of liquid through some other route than the digestive track. The compositions can take such forms as suspensions, solutions or emulsions in oily or aqueous vehicles, and can contain formulatory agents such as suspending, stabilizing and/or dispersing agents. Suitable pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

In one embodiment, the plasma or serum fraction is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to injection.

The plasma or serum fraction can be formulated as neutral or salt forms. Pharmaceutically acceptable salts include those formed with anions such as those derived from hydrochloric, phosphoric, acetic, oxalic, taratic acids, etc., and those formed with cations such as those derived from sodium, potassium, ammonium, calcium, ferric hydroxides, isopropylamine, triethylamine, 2-ethylamino ethanol, histidine, procaine, etc.

For parenteral administration, in one embodiment, the plasma or serum fraction composition of the present invention is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the plasma or serum fraction uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g. polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The parenteral form of the present invention can be delivered by injection. Formulations for injection can be presented in unit dosage form, e.g., in ampoules or in multi-dose containers, with an added preservative. In one embodiment of the present invention, the composition is delivered by bolus injection. In another embodiment, the plasma cocktail can be administered continuously. Continuous administration may be performed according to any of the methods and techniques known to those skilled in the art, including continuous injection or infusion using a surgically implantable pump, such as an Arrow Model 3000 (Arrow International, Walpole, Mass.) or MiniMed 2001 (MiniMed Techologies, Sylmar, Calif.); injection or infusion using an externally worn pump, such as the MiniMed 504S (MiniMed Technologies) or the H-Tron V100 (Disetronic Medical Systems, Minnetonka, Minn.); or difflusion from a biologically-derived bio-resorbable hydrogel, such as a chitosan hydrogel or N,O-carboxymethyl chitosan (NOC-chitosan) hydrogel, alone or in combination with a charged or uncharged polymeic agent to control hydrogel porosity and/or hydrogel stability and/or kinetics of protein release, such as polylysine or polypropylene glycol; or diffusion from a synthetically-derived bio-resorbable hydrogel, such as polypropylene glycol, alone or in combination with a charged or uncharged polymeic agent to control hydrogel porosity and/or hydrogel stability and/or kinetics of protein relaease, such as polylysine.

A particular mode of administration of the active compound is through subcutaneous injection, which can optionally include an inert diluent or carrier. Representative carriers are water, physiological saline, phosphate buffered saline (PBS), dextrose solution, or Ringer's Solution.

The plasma or serum fraction can be also be lyophilized (i.e., dried and the water evaporated) and will generally include an inert diluent or edible carrier. The lyophilized composition can be reconstituted with liquid prior to use. Alternatively, it may be enclosed in gelatin capsules or compressed into tablets.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). Other non-limiting examples of suitable pharmaceutical exipients include glucose, sucrose gelatin, malt, rice, flower, chalk, silica gel, sodium stearate, glycerol monostearate, sodium chloride, dried skim milk, glycerol, water, ethanol and the like. The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

If administered by nasal aerosol or by inhalation, the plasma or serum fraction composition is prepared according to techniques well-known in the art of pharmaceutical formulation and may be prepared as solutions in saline, employing benzyl alcohol or other suitable preservatives, absorption promoters to enhance bioavailability, fluorocarbond, and/or other solubilizing or dispersing agents known in the art.

If rectally or vaginally administered in the form of suppositories, the plasma or serum fraction may be prepared by mixing the drug with a suitable non-initiating excipient, such as cocoa butter, synthetic glyceride esters of polyethylene glycols, which are solid at ordinary temperatures, but liquefy and/or dissolve in the rectal cavity to release the drug.

The plasma or serum fraction can also be mixed with other active materials that do not impair the desired action, or with materials that supplement the desired action, such as antibiotics, anti-fungals, anti-inflammatories, protease inhibitors, or other nucleoside or non-nucleoside antiviral agents, as discussed in more detail above. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

The plasma or serum fraction is included in the pharmaceutically acceptable carrier or diluent in an amount sufficient to deliver to a patient a therapeutically effective amount to lower detectable viral load and/or lower risk of infection, whether opportunistic or with HIV, without causing serious side effects in the treated patient. It is possible to measure viral load through one of many methods well known in the art, and resistance to infection can also be measured in vitro by methods well known in the art. If administered by nasal aerosol or inhalation, the plasma or serum fraction is prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The plasma or serum fraction may be administered at once, or may be divided into a number of smaller doses to be administered at varying intervals of time.

### EXAMPLES

### Example 1

### Production of the Immunogen

Patients who are HIV positive and with a detectable viral load, and preferably with a viral load above 2,000, are used for blood samples because this provides a sufficient challenge to the animal's immune system to generate a response. The blood taken from the patient is centrifuged at 32,000 rpm at room temperature using standard sterile laboratory techniques and the resulting patient plasma/serum is frozen at −20° C. Preferably the sample is frozen no more than 24 hours, but may be left for longer.

### Example 2

### Inoculation of the Animal

The animal used in the process is first inspected by a veterinarian and evaluated for any underlying abnormalities in the animal and for any pathogens that may cause a possible zoonosis. Once the animal is found to be healthy it is then well maintained in a clean environment and monitored by a veterinarian on a regular basis.

The HIV positive patient plasma sample prepared as outlined in Example 1 is allowed to thaw to room temperature and approximately 3cc of the patient plasma/serum is injected subcutaneously into the animal according to standard sterile procedures. Once the animal has been injected with the sample, the animal is carefully marked and labeled assigning it a number and indicating where the sample was taken and from whom. The animal is then allowed approximately a three week period before harvesting any of its blood.

### Example 3

### Preparation of the Plasma or Serum Sample

Once the three week period has passed, the specimen animal is injected with 0.5 cc rompun (for ease of handling). After the animal has reached the appropriate level of anesthesia, the external jugular area is sterilely prepped and draped. An 18 gauge one inch needle attached to a 60 cc lure lock syringe is introduced into the external jugular vein and approximately 200 to 400 cc total (using 4-8 lure lock syringes) is sterilely extracted. The blood is immediately transferred to an ice bath to keep it cool, immediately following which the blood is centrifuged with an office model centrifuge at 32,000×g room temperature and the resultant plasma/serum mixture is sterilely removed and passed through a 0.5 micron suction filtration device. The sterile product is then placed in an ice bath when transportation to main laboratory facilities is necessary, where the product is sterilely filtered through a 0.2 micron suction filtration. It is then transferred to a non-refrigerated ultracentrifuge for twenty minutes at 90,000×g after which the supernatant is sterilely transferred to appropriately sized tubes for a refrigerated ultracentrifuge and spun at 150,000×g for twenty minutes. The supernatant is then passed through an anhydrous filter and then passed through a 0.1 micron suction filtration and into sterile containers where it is then placed into a −70° C. freezer and kept for at least 48 hours. After the 48 hours have passed, samples of the batch are taken and cultured both anaerobic ally and aerobically for any pathogens. Once the culture gives negative results, the plasma or serum sample is ready for further processing.

### Example 4

### Antibody Depletion

Antibody depletion experiments were conducted on the product prepared as described in Example 3 by the panning technique as follows. Experiments were completed to remove IgG, IgM and both IgG and IgM. Two wells each of a polystyrene high protein-binding flat-bottomed plate were coated with 100 μL of 1 μg/mL of anti-goat IgG, anti-goat IgM, anti-goat IgG+anti-goat IgM antibodies, or PBS. The plate was incubated for 3 hours at room temperature. Following the incubation, the antibodies and PBS were removed and 200 μL of blocking solution (0.05% Tween 20, 10 mg/mL BSA in PBS) was added and the plate was incubated overnight at 4° C. Following the overnight incubation, the wells were washed 5 times with wash buffer (PBS plus 0.05% Tween-20). One hundred fifty microliters (150 μL) of the serum product (designated VR-30) was added to each of the wells and the plate was incubated for 2 hours at 37° C.

The serum was then removed and used in the HIV-β-galactosidase attachment assay. The depleted material was evaluated for activity in parallel with undepleted VR-30 (VR-30 Std Treatment) and mock-depleted VR-30 (VR-30 PBS). In all cases, complete anti-HIV activity was observed following the treatments indicating that the removal of goat immunoglobulins (IgG, IgM and IgG+IgM) did not impact the anti-HIV activity of the test material. The results of this experiment are shown in Table I, below.

TABLE I
Antibody Depletion of VR-30

| | IC50 | TC50 | |
|---|---|---|---|
| COMPOUND | (DILUTION) | (DILUTION) | Therapeutic Index |
| Chicago Sky Blue | 1.3 | >10 | >7.7 |
| (μg/mL) | | | |
| Dextran Sulfate | 0.65 | >25 | >38.5 |
| (μg/mL) | | | |
| T.V. (IgG) | ˜1:25 | >1:10 | — |
| T.V. (IgM) | ˜1:30 | >1:10 | — |
| T.V. (IgG + IgM) | ˜1:20 | >1:10 | — |
| T.V. (PBS) | ˜1:33 | >1:10 | — |
| T.V. | ˜1:20 | >1:10 | — |
| (Std Treatment) | | | |

Preliminary heat denaturation further evidenced that the active component(s) is not an antibody. Incubation of VR-30 at 56° C. for 30 min resulted in inactivation of specific anti-HIV activity, while the same treatment of control goat serum did not reduce the level of an observed non-specific inhibition (data not shown).

### Example 5

### Anti-HIV Activity of the Initial Plasma or Serum Sample

The anti-HIV activity of the initial plasma or serum sample is used as a baseline for determining the efficiency of the concentration of the anti-HIV activity for the fractionation procedures described below.

The anti-HIV-activity of the initial serum sample is determined by: (1) measuring the protein concentration of the serum fraction using Bradford or Lowery based method for determining protein concentration; (2) determining the activity of the fraction using established in vitro anti-viral assay (attachment assay); (3) assigning a unit of activity to the fraction based on the amount of protein needed to achieve a 50% inhibition of viral replication/infectivity (IC50). This unit can be used as a baseline to track the fold purification obtained through the fractionation process.

### Example 6

### Ammonium Sulfate Precipitation

Immunoglobulin G (IgG) is depleted from the sample using a 33% ammonium sulfate precipitation. With the serum sample kept on ice and with constant slow stirring, a saturated ammonium sulfate solution (e.g., 450 g of ammonium sulfate in water to 500 mL) is added to 33% v/v (1 ml saturated ammonium sulfate per 2 mL of serum). The sample is allowed to stir on ice for a period of time ranging from approximately 2 to approximately 4 hours, and then centrifuged for approximately 12,000×g for approximately 20 minutes at 4° C. The supernatant is carefully removed to a clean tube. The precipitate or pellet should contain the majority of the IgG.

The pellet is then washed twice with a volume of ice cold 33% ammonium sulfate solution equivalent to the original volume of the fraction, and then centrifuged at approximately 12,000×g for 20 minutes at 4° C. for each wash. The pellet is then dissolved in a volume of ice cold buffer A equivalent to 10% of the starting volume. The buffer should be suitable for in vitro assays and down stream purification procedures. The suspended pellet is then desalted using a desalting column or dialyzed overnight in ice cold buffer at 4° C. in order to remove any ammonium sulfate.

If a desalting column is used, the procedure involves decanting buffer from the top of column, and loading the sample onto the column. Then, 10 ml of Buffer A is applied to the top of the column and allow to flow through the column. The sample (no more than 3 mL) should be applied to the top of the column and allowed to pass through the column by gravity flow. Fractions of 0.5 ml volume into siliconized tubes.

Then, the 33% ammonium sulfate supernatant produced is fractionated with a 66% ammonium sulfate precipitation. The concentration of the supernatant is adjusted to 66% by adding 1 ml of saturated ammonium sulfate for every 1 ml of supernatant. The 66% precipitation is then performed as above with respect to the 33% precipitation to provide a 66% pellet. The 66% pellet is then washed twice with a volume of ice cold 66% ammonium sulfate solution equivalent to the original volume of the fraction, and then centrifuged at approximately 12,000 x g for 20 min 4° C. for each wash. The pellet is then dissolved in a volume of ice cold buffer A to 10% of the original fraction volume. The suspended pellet is desalted using either a desalting column or by dialysis against buffer A at 4° C. overnight to remove any ammonium sulfate.

Next, the activity of each fraction is tested. The protein concentration of each fraction is measured using the BioRad protein assay. The viral attachment assay is performed using a concentration of protein equivalent to or less than the amount of protein from the unfractionated material that yielded 50% inhibition in the initial assay. A unit of activity is assigned to the fraction based on the amount of protein and the initial dilution needed to achieve a 50% inhibition of viral replication/infectivity (IC50). The fold purification is determined by calculating the ratio of the activity of the purified material to the activity of the starting material. This unit should be calculated as activity per mass of total protein and should increase as the purification process is applied.

An analysis of the active fraction or fractions is then performed. Native and denatured PAGE is performed to determine the approximate number and sizes of the proteins in the active fraction(s). An immunoblot analysis is performed to test for the presence of immunoglobulins and albumin in the active fraction.

### Example 7

### DEA Affi-Blue Column Chromatography

DEAE Blue Econo-Pac cartridges (BioRad) are used for initial fractionation of the serum sample. These reagents contain an affinity matrix of Cibacron blue dye coupled with a DEAE anion exchanger. The Cibacron blue dye has a high affinity for protein albumins and the DEAE allows for the separation of proteins based on their charge. Immunoglobulins do not bind to the Cibacron blue dye, albumins will bind very tightly, and other proteins should have low to intermediate binding capacity. The various proteins within the serum will also have a range of DEAE binding capacities. The low to intermediate Cibacron blue and the DEAE binding proteins can be eluted from the matrix using competing salt ions. Elution can be performed using a step gradient (as outlined below) or with a linear gradient. The procedure outlined herein details the use of a syringe to load the protein and buffers onto the column; however, the procedure may also be adapted for use with a low or high pressure chromatography system and a larger chromatography column.

(i) Preparation of Buffers

The various buffers needed are prepared. The equilibration and wash buffer (“Buffer A”) contains 28 mM NaCl and 20 mM Tris-HCl pH 8.0. The elution buffers (“Buffer E”) for the DEAE blue cartridge include E1, E2, E5 and E14. E1 is 100 mM NaCl, 20 mM Tris-HCl pH8.0; E2 is 250 mM NaCl 20 mM Tris-HCl pH 8.0; E5 is 500 mM NaCl 20 mM Tris-HCl pH 8.0; E14 is 1.4M NaCl 20 mM Tris-HCl, pH 8.0. Regeneration buffer 1 (Buffer G) is 1.4 M NaCl 100 mM Acetic Acid pH 3.0 40% Isopropanol. Regeneration buffer 2 (“Buffer I”) is 28 mM NaCl 20 mM Tris-HCl 2M Guanidine-HCl. Storage Buffer for DEAE Blue cartridge is 20 mM Sodium Phosphate pH 7.5 0.02% Sodium Azide. Necessary additives for protein stabilization or activity (such as protease inhibitors, glycerol, EDTA, DTT, etc.) may be added to any of the buffers as deemed necessary. The pH listed for the buffers should be the pH at 25° C. All buffers should be chilled to 0-8° C. prior to use to minimize loss of anti-viral activity.

(ii) Sample Preparation

The sample should be in Buffer A. If the starting material is not already in Buffer A, equilibrate the sample with buffer A using a desalting colunm or by dialysis.

(iii) Preparing the Cartridge for Use

The cartridge is prepared for use by washing it with 10 ml of Buffer G at a flow rate of 1 ml/min to remove any residual dye. It is then washed with 5 ml of buffer E14 at a flow rate of 2 ml/min. A small amount of air may remain just above the upper frit and in the inlet nozzle of the cartridge. The cartridge should be inverted so that the arrow points upward, allowing air to be expelled into the cartridge and out through the outlet nozzle. The cartridge is then washed with Buffer A for 10 minutes at a flow rate of 2.0 ml/min. The cartridge should then be equilibrated with Buffer A for 2 min at 1.0 ml/min. The cartridge should then be inverted, so it points downward.

(iv) Chromatography

The following procedures are used when a syringe is used to load and elute the protein onto the column. (A chromatography system utilizes the same buffers, but they are applied with the system pump and the protein is eluted with a linear salt gradient rather than a step gradient). To load the sample, a sterile syringe is pre-wet with Buffer A by sucking and expelling buffer into and out of the syringe. The plunger is removed from the syringe and attached to the cartridge using a luer lock connector. The equilibrated sample is then added to the barrel of the syringe. The plunger is inserted, and the sample is pushed through the cartridge taking care not to inject air into the cartridge. The flow through collected into a clean siliconized tube as the sample is being loaded. The flow through can be collected into more than one tube.

The cartridge is then washed. The syringe is removed from the cartridge, and washed with buffer A. The plunger is pulled from the syringe and re-attached the barrel to the cartridge. The barrel is then filled with Buffer A and pushed through the cartridge at a flow rate of about 1 ml/min. Wash fractions of ‘0.5 ml are then collected in siliconized tubes. Wash with a total volume equivalent to 3-5 times the cartridge volume.

The bound proteins are then eluted with a step gradient. The syringe is removed from the cartridge and washed with Buffer E1. The syringe is then attached to the cartridge and pushed through 10 to 15 ml of buffer E1. The fractions are collected in siliconized tubes. The elution steps are then repeated with E-Buffer's of increasing NaCl concentrations. Alternatively, a linear salt gradient from 0 to 500 mM NaCl can be used to elute the bound serum proteins.

The sample is then analyzed. The concentration of protein in each sample is analyzed using the Bio-Rad Protein assay or similar. The peak fractions are analyzed for anti-HIV activity using the attachment assay. The protein profile of the fractions is analyzed on a polyacrylamide gel. The presence of IgG and IgM by are detected by western blot.

### Example 8

### Protein G Affinity Chromatography

Protein G is a cell surface protein of group G streptococci. It is a Type III F_{c} receptor that binds to the F_{c} region of IgG by a non-immune mechanism. Protein G binds tightly to different subclasses of IgG from a variety of species including human, rabbit, horse, sheep, and goat. IgG immunoglobulins are removed from active fractions using Protein G coupled to a Sepharose matrix. The IgG binds tightly to the matrix and the active non-immunoglobulin fraction will not bind and will be collected in the flow though during application.

An Amersham HiTrap Protein G HP column with a syringe for application (or high-low pressure chromatography system) of the sample and buffer can be used for Protein G affinity chromatography. The buffers are prepared, including a binding buffer (20 mM Sodium Phosphate, pH 7.0) and an elution buffer (0.1 M Glycine-HCl, pH 2.7). Necessary additives for protein stabilization or activity (such as protease inhibitors, glycerol, EDTA, DTT, etc.) may be added to any of the buffers as deemed necessary. The pH listed for the buffers should be the pH at 25° C. All buffers should be chilled to 0-8° C. prior to use to minimize loss of anti-viral activity.

The sample is prepared by adjusting to the composition of Protein G binding buffer using either a desalting column or by dialysis. Use of a desalting column involves decanting buffer from the top of column, and loading the sample onto the column. Then, 10 ml of Binding Buffer is applied to the top of the column and allow to flow through the column. No more than 3 ml sample should be applied to the top of the column and allowed to pass through the column by gravity flow. Fractions of 0.5 ml volume into siliconized tubes.

If the sample is cloudy or viscous, the buffer adjusted sample is passed through a 0.45 μm filter prior to loading. The column is then prepared. Silicon collection tubes are prepared for the collection of eluted IgG by adding 60-200 1 of 1M Tris-HCl pH 9.0 per ml of fraction collected. Tris should not be added to tubes for collection of flow through material. Using a syringe or pump, the column is washed with 10 column volumes of binding buffer.

The sample is then applied to the column and the flow through is collected in siliconized tubes. The flow through will contain the non-IgG proteins. The flow through is collected in 0.5 to 1 mL fractions. The column is washed with 5-10 column volumes of binding buffer. The washed fractions are then collected in siliconized tubes. The bound IgG is then eluted with 2-5 column volumes of elution buffer. The eluted protein material is collected in siliconized tubes containing 1M Tris-HCl, pH 9.0.

The sample is then analyzed. The protein concentration in each sample is quantified using the Bio-Rad Protein assay or similar. The peak fractions are analyzed for anti-HIV activity using the attachment assay. The protein profile of the fractions is analyzed on a polyacrylamide gel. The fractions are also analyzed for the presence of IgG and IgM by western blot.

### Example 9

### Gel Filtration Chromatography

Gel filtration chromatography is a method of separating molecules based on their size. This procedure may be used at any step in the purification process. Immunoglobulins and serum albumins will fractionate with the higher molecular weight proteins (in the first eluted fractions to come off of the column) and lower molecular weight proteins, such as cytokines, will come off of the column in the latter fractions. Different gel filtration media can be used for separation purposes depending on the size of the protein to be isolated.

The gel filtration media can be Sephacryl S100 or S200 HR. The sample is prepared by adjusting the composition of the gel filtration buffer using either a desalting column or by dialysis. The desalting protocol involves decanting buffer from the top of column, and loading the sample onto the column. Then, 10 ml of the gel filtration buffer is applied to the top of the column and allow to flow through the column. No more than 3 ml sample should be applied to the top of the column and allowed to pass through the column by gravity flow. Fractions of 0.5 ml volume into siliconized tubes. If necessary, the sample can be concentrated using a Centricon concentrator (or comparable). If the sample is cloudy or viscous, the buffer-adjusted sample is passed through a 0.45 μm filter prior to loading.

Using a chromatography system, the column is properly attached to the system and equilibrated with gel filtration buffer. The sample is loaded on to the column through the sample loop. Proteins are then eluted with gel filtration buffer at a constant flow rate. The fractions are collected in siliconized tubes.

The samples are analyzed for anti-HIV activity using the attachment assay. The samples are further analyzed for protein profile on a polyacrylamide gel. The samples are also analyzed for the presence of IgG and IgM by western blot.

### Example 10

### Preparation of the Product

Pathogen-free goats inoculated with plasma from an uninfected human donor, and plasma from an HIV-infected human donor. Blood was extracted from the goats just prior to inoculation (Week 0) and at weekly intervals up to 5 weeks post inoculation. Serum was prepared from the goat blood and the anti-HIV activity of the serum samples was tested using the HIV-β-galactosidase attachment bioassay. The amount of anti-viral activity produced in individual goats varied. The results are shown in FIG. 1. Four of the six animals inoculated with plasma from the HIV-infected donor had increases in anti-HIV activity present in serum that peaked on week 3 post inoculation. The three week window is too short of a time period for the generation of a significant humoral response to a single inoculation with a non-replicating virus.

### Example 11

### Partial Fractionation of the Serum Product

Anti-HIV active goat serum was collected from animal number 26 (shown in FIG. 1) inoculated as described in Example 10. The serum was equilibrated with Buffer A (10 mM Tris-HCl pH 8.0, 28 mM NaCl) using a Bio Rad DG-10 gravity flow column at 4° C. Fractions of approximately 1 ml each were collected by hand and analyzed for protein content using the Bio Rad Protein Assay with BSA as a reference standard. The elution profile from the desalting column is shown in FIG. 2.

The fractions for milliliters 4 through 8 from the DG-10 column were pooled and subjected to DEAE-blue chromatography using a Bio Rad 5 ml DEAE-blue cartridge and 20 ml syringe. Sample was loaded onto the cartridge with the syringe and the cartridge was washed with 25 ml of ice cold buffer A (fractions 1 to 35), followed by sequential washing with ice cold buffer A containing 0.1M NaCl (fractions 36 to 50), ice cold buffer A containing 0.5 M NaCl (fractions 51 to 66), and a final elution with ice cold buffer A containing 1.4 M NaCl (fractions 67 to 77). All fractions were collected manually and immediately placed on ice. The protein concentration in each fraction was determined using the Bio Rad Protein assay.

Selected fractions were subjected to SDS-PAGE on 8-16% Bio Rad Criterion gels followed by Coomassie blue staining (FIG. 3) and immunoblot analysis with horseradish peroxidase conjugated rabbit-anti-goat IgG polyclonal antisera (FIG. 4). The following amounts of total protein were loaded onto the gels for both the Coomassie stain and immunoblot gels, 20 μg of total protein for Week 0 serum, Week 3 serum, and DG-10 column fractions (pool, DG-1, DG-4, and DG-5); 10 μg of total protein for DEAE-blue fractions 12, 47, 57, and 68; 5 tg of total protein for DEAE-blue fraction 35; 2.5 μg of total protein for DEAE-blue fraction 77; and 2 μg of total purified total goat IgG (NIH AIDS Research and Reference Reagent Program). Proteins for immunoblot analysis were transferred to 0.45 micron nitrocellulose using a Bio Rad semi-dry transfer apparatus. The membrane was blocked overnight at 4° C. with 5% nonfat milk in PBS-T (PBS plus 0.1% Tween-20) and probed with a 1:2000 dilution of horse radish peroxidase conjugated rabbit-anti-goat IgG polyclonal antibody in PBS-T plus 1% nonfat milk for 1.5 hours at room temperature. Following probing, the membrane was washed extensively with PBS-T and developed with One-step TMB HRP-detection reagent (Pierce) according to the manufacturers instructions.

Selected fractions were also analyzed for antiviral activity using the HIV 0-galactosidase attachment assay as shown in Table II.

TABLE II

| | | | | Fold increase |
|---|---|---|---|---|
| Fraction | EC50 (mg/ml) | TC50 (mg/ml) | TI | in activity |
| Chicago Sky | 0.10 | >10 | >100 | ND |
| Blue | | | | |
| Week 0 | 1.25 | >2.5 | >2.0 | 1.00 |
| Week 3 | 0.58 | >2.5 | >4.3 | 2.14 |
| DG-10 Pool | 0.85 | >2.5 | >2.9 | 1.46 |
| DG-4 | 0.06 | >2.5 | >1.3 | 20.66 |
| DEAE-blue 12 | 1.98 | >2.5 | >1.1 | 0.62 |
| DEAE-blue 47 | 2.37 | >2.5 | >42 | 0.52 |
| DEAE-blue 57 | >2.5 | >2.5 | >2.9 | 0.50 |
| DEAE blue 68 | 1.98 | >2.5 | >1.0 | 0.52 |

Each fraction was adjusted to a 2× high test concentration of 5 mg/ml total protein and 6×½-log serial dilutions prepared in buffer A. The diluted fractions were assessed in triplicate for anti-HIV activity in the HIV β-galactosidase attachment assay and the EC₅₀ calculated as the amount of protein necessary for 50% inhibition of HIV attachment and replication relative to a no compound control. The 50% cellular toxicity (TC₅₀) was determined for each fraction in parallel plates using a tetrazolium dye cell viability (XTT) end point assay. The fold increase in activity was calculated as a relative decrease in EC₅₀ to the Week 0 material. Chicago Sky Blue was included as a positive control for inhibition of viral attachment.

Analysis of the DEAE-blue cartridge fractions revealed that the majority of IgG was present in the unbound fractions and anti-HIV activity was not enriched in any of the fractions recovered from the column. This included fractions that were enriched for IgG and presumably albumin (fractions 12 and 68, respectively, FIGS. 3 and 4, and Table II). However, a later fraction (corresponding to milliliter 7, FIG. 2) from the DG10 desalting column was greater than 20 times more active than a Week 0 serum sample and greater than 10 times more active than the initial starting material (DG-4, FIG. 3 and Table II). Since the desalting works on the principle of gel exclusion chromatography, it would be expected that later fractions eluting from the column would contain a larger proportion of lower molecular weight proteins. Visual examination of the proteins present in the Coomassie stained gel for the DG-4 fraction reveal an increase in the intensity of protein bands between the molecular weights of 29 and 53 kilodaltons. These results further confirm that the active component is not IgG and indicate that the active anti-HIV component is likely a lower molecular weight protein.

### Example 12

### Partial Fractionation of the Serum Sample

A total of 88 mls of serum from the bleed out of animal number 26 in Study Number 5 100-01-05 was removed from the −80 freezer and allowed to thaw on ice.

(i) Ammonium Sulfate Precipitation

The serum was divided into 4 polypropylene centrifuge tubes each containing a flea stir bar and placed in an ice bath. A 33% ammonium sulfate precipitation was performed by adding one part of ice cold saturated ammonium sulfate solution per 2 parts serum in 0.5 ml aliquots to each tube with constant stirring. The mixture was allowed to stir on ice for 1.5 hr. Following the 1.5 hrs, the mixture was subjected to centrifugation at 12,000×g for 20 minutes 4° C. The supernatant was transferred to a 400 ml polypropylene beaker and subjected to 66% ammonium sulfate precipitation, as above, with the addition of 1 part saturated ammonium sulfate solution per 1 part supernatant. The pellets from the 33% ammonium sulfate precipitation were washed with 33% ammonium sulfate in PBS and then suspended in 20 mls PBS. The supernatant from the 66% ammonium sulfate precipitation was decanted into 50 ml conical tubes and the pellet was suspended in 20 ml PBS.

The results of the precipitation procedure are shown in Table III below:

TABLE III

| | | | Total Protein | |
|---|---|---|---|---|
| Fraction | Volume (ml) | Conc. (mg/ml) | (mg) | % Protein |
| Initial | 88 | 79.85 | 7026.6 | 100 |
| Bleed | | | | |
| Out | | | | |
| 33% | 81 | 40.12 | 325 | 4.62 |
| Wash | | | | |
| 33% | 23 | 38.45 | 884.3 | 12.58 |
| Pellet | | | | |
| 66% | 39.6 | 88.46 | 3503.1 | 49.85 |
| Pellet | | | | |
| 66% | 193.2 | 43.81 | 846.5 | 12.05 |
| Sup. | | | | |

(ii) DEA-Blue Column Chromatography

Two milliliters of a partially fractionated serum sample previously subjected to 66% 5 ammonium sulfate precipitation and dialysis was further fractionated on a 5 ml BioRad DEAE-Blue column using a BioRad Biologic LP chromatography system.

A DEAE-Blue Econo column (BioRad) was prepared according to the manufacturer's instruction. Briefly, using a syringe, the column was washed with 10 ml of freshly prepared Buffer R (1.4 M NaCl, 0.1M Acetic acid, 40% Isopropanol) followed by washings with 5 ml Buffer B (1.4 M NaCl, 20 mM Tris-HCl pH 7.5, 10% Glycerol) and 30 ml of Buffer A (28 mM NaCl, 20 mM Tris-HCl pH 7.5, 10% Glycerol). The column was then attached to the BioRad BioLogic LP Chromatography system and equilibrated with Buffer A (approximately 50 ml at 1 ml/mn). Two milliliters of a dialyzed 66% ammonium sulfate pellet fraction prepared above was thawed on ice and loaded onto the BioLogic LP using a 3 ml syringe and a 2 ml sample loop. The sample was loaded onto the column by running 6 mls of Buffer A at a flow rate of 1 ml/min through the sample loop, and the column was washed with eight column volumes (40 ml) of Buffer A at the same flow rate. The bound proteins were eluted using a linear salt gradient from 28 mM NaCl to 1.4 mM NaCl over 40 mls at 1 ml/min. Eighty two fractions of approximately 0.75 ml each were collected into siliconized 1.5 ml microcentrifuge tubes. Chromatography was monitored using the LP Data View software package. All fractions were stored at −80 C until analyzed. Samples were analyzed for protein content using the BioRad Protein assay kit with BSA as a quantitative standard. The majority of protein was bound to the column and eluted with the linear gradient; a minority of protein came through in the unbound flow through.

The chromatographic profile for the DEAE-Blue column fractionation of the 66% ammonium sulfate pellet is shown in FIG. 5. Detection of protein occurred four minutes into the run at fraction 6. The concentration of protein in the flow through fractions peaked between 9 and 10 minutes into the run (fraction 13) and then declined until around 26 minutes (fraction 36). The majority of protein in the flow through fractions (concentrations greater than 100 ng/μl) was contained in fractions 9 through 20. A linear salt gradient was initiated 40 minutes into the run and the majority of total protein was eluted between 40 and 54 minutes (fractions 56 to 74). Based on the chromatographic profile, this gradient did not appear to selectively fractionate proteins based on ionic charge.

(iii) SDS-PAGE and HIV Attachment Assay

Selected fractions were also analyzed by SDS-PAGE and anti-viral activity in an HIV attachment assay.

SDS-PAGE: Five micrograms of total protein from selected fractions, including the initial bleed out serum, the dialyzed 66% ammonium sulfate pellet, and DEAE-blue fractions 11-15, and 57-77, were prepared in 2× Lammeli sample buffer, heated to 95° C. for 5 min, and loaded onto a Criterion Precast 8-16% polyacrylamide Tris-HCl gel (Bio Rad). The gel was also loaded with Bio Rad prestained broad range molecular weight markers. The proteins were electrophoresed at 100 V until the bomophenol dye in the sample buffer reached the bottom of the gel. The gel was subjected to Coomassie blue staining with BioSafe Coomassie G250 stain (Bio Rad) according to the manufacturers recommended method. An image of the stained gel was captured on an Alphalmager 2000 and the supporting software was used to calculate molecular weight of the fractionated proteins.

SDS-PAGE analysis and Commassie staining revealed five proteins that appeared to be enriched in the unbound fractions. As indicated by the arrows in FIG. 6, five proteins appear to be enriched in fractions 11 through 15, although other proteins may be present that are not detected by the staining technique. These proteins are (from the top) 49.1, 30.1, 28.6, 14.1, and 12.2 kDa in molecular weight. The presence of light staining bands of equivalent size in fractions 57, 59, and 61, but not in fractions 63 through 77, may suggest that these proteins are the active component. The identity of the other three enriched protein bands can not be extrapolated from the data. It remains possible that the active component is not visible in this analysis.

HIV Attachment Assay: A crude serum sample dialyzed against Buffer A, the dialyzed 66% ammonium sulfate pellet, and DEAE-Blue fractions 12, 14, 60, 62, 64, and 66 were analyzed for anti-HIV activity in a virus attachment assay. These fractions were diluted in Buffer A to a 2× high test concentration and analyzed for anti-HIV activity in a cell attachment assay that utilizes a galactosidase reporter to measure virus attachment and infection. Sister plates were used to evaluate fraction toxicity using an XTT based end point. Briefly, on the day prior to compound and virus addition, 100 μL of HeLa LTR β-Gal cells at a density of 1×10⁴/well in 10% complete DMEM were plated in 96-well flat bottomed plates and incubated overnight at 37° C./5% CO₂. Duplicate plates were seeded in order to evaluate efficacy and toxicity in parallel. Following the overnight incubation, selected protein fractions diluted to a high test concentration in culture media and were further diluted in ½ log increments such that a total of 6 concentrations were evaluated. The high test concentrations for each fraction are displayed in Table IV.

TABLE IV

| Tube | Fractions | 2X Concentration | | High Test |
|---|---|---|---|---|
| 1 | 66% dialyzed pellet | 20 | mg/ml | 10 mg/ml |
| 2 | DEAF-Blue #12 | 0.25 | mg/ml | 0.125 mg/ml |
| 3 | DEAF-Blue #14 | 0.25 | mg/ml | 0.125 mg/ml |
| 4 | DEAF-Blue #60 | 1.5 | mg/ml | 0.75 mg/ml |
| 5 | DEAF-Blue #62 | 2.5 | mg/ml | 1.25 mg/ml |
| 6 | DEAF-Blue #64 | 2.5 | mg/ml | 1.25 mg/ml |
| 7 | DEAF-Blue #66 | 2.5 | mg.ml | 1.25 mg/ml |
| 8 | Dialyzed Serum | 20 | mg.ml | 10 mg/ml |

The results in Table IV show high test concentration of each fraction in attachment and toxicity assays.

Medium was removed and 50 μL of the prepared compound dilutions were added in triplicate to both plates. A volume of 50 μL of 10% complete DMEM was added to the virus control wells and 100 μL was added to the cell control wells. Plates were incubated for approximately 15-30 minutes while virus (HIV-1 IIIB) was being prepared. The appropriate titer of virus used in the assay was determined and prepared and 50 μL was then added to the wells containing compound and the virus control wells on the efficacy plate. On the toxicity plate, 50 μL of 10% complete DMEM was added to these same wells. Plates were incubated for 2 hours at 37° C./5% CO₂. Following the incubation, the cell monolayers were washed three times with 150 μL of RPMI-1640 media without additives at room temperature. Following the washing steps, 200 μL of 10% complete DMEM was added to all wells and the plates were incubated at 37° C./5% CO₂ for 48 hours. The toxicity plates were evaluated by the addition of the tetrazolium dye XTT which is converted by mitochondrial enzymes in viable cells to a soluble formazan product. Efficacy plates were evaluated by the chemilluminescence detection assay.

The results are shown in FIG. 6 and Table V (below). The reported EC₅₀ in FIG. 6 for fractions 59, 61, 63 and 65 are those for the flanking fractions 60, 62, 64, and 66, respectively. All EC₅₀'s in FIG. 2 and Table IV are the concentration of total protein (μg/ml) in a given fraction that inhibited viral replication in the attachment assay to 50% of the no compound control. The amounts reported for fractions 12, 14, and 60 are the lowest dilution tested for these fractions which completely inhibited viral replication in these assays.

TABLE V

| | | | | Fold Increase in |
|---|---|---|---|---|
| | | | | Relative Activity |
| | | | | (Dialyzed |
| | | | Therapeutic | Serum/66% |
| Sample | EC50 | TC50 | Index | Pellet |
| Chicago Sky Blue | 0.6 | >10 | >17 | NA |
| (ug/ml) | | | | |
| 66% Dialyzed | 85 | >10 | >118 | 3.53/1.00 |
| Pellet (ug/mL) | | | | |
| DEAE Blue #12 | <0.39 | >125 | >320 | 769/218 |
| (ug/mL) | | | | |
| DEAE Blue #14 | <0.39 | >125 | >320 | 769/218 |
| (ug/mL) | | | | |
| DEAE Blue #60 | <2.3 | >750 | >320 | 130/37 |
| (ug/mL) | | | | |
| DEAE Blue #62 | >1250 | >1250 | — | 0.24/0.07 |
| (ug/mL) | | | | |
| DEAE Blue #64 | >1250 | >1250 | — | 0.24/0.07 |
| (ug/mL) | | | | |
| DEAE Blue | >1250 | >1250 | — | 0.24/0.07 |
| #66(ug/mL) | | | | |
| Dialyzed Serum | 300 | >10 | >33 | 1.00/0.28 |
| (ug/mL) | | | | |

The analysis revealed that the unbound protein fractions were greater than 218 times more active than the starting material in inhibiting HIV attachment in a β-galactosidase reporter assay. With the exception of one fraction, the bound proteins were only 0.06 times as active as the starting material.

The invention has been described with reference to its preferred embodiments. Variations and modifications of the invention will be obvious to those skilled in the art from the forgoing detailed description of the invention.

What is claimed is:

1. A plasma or serum fraction derived from a mammal exposed to an inoculant, which fraction has been depleted of two or more high molecular weight proteins.

2. The plasma or serum fraction of claim 1, wherein the fraction has been depleted of immunoglobulins and albumin.

3. The plasma or serum fraction of claim 1, wherein the fraction has been depleted of from about 1 to about 100% of the two or more high molecular weight proteins.

4. The plasma or serum fraction of claim 3, wherein the fraction has been depleted of from about 20% to about 100% of the two or more high molecular weight proteins.

5. The plasma or serum fraction of claim 3, wherein the fraction has been depleted of from about 50% to about 100% of the two or more high molecular weight proteins.

6. The plasma or serum fraction of claim 3, wherein the fraction has been depleted of from about 75% to about 100% of the two or more high molecular weight proteins.

7. The plasma or serum fraction of claim 1, wherein the inoculant is a viral inoculant.

8. The plasma or serum fraction of claim 7, wherein the viral inoculant is an HIV-bearing inoculant.

9. The plasma or serum fraction of claim 7, wherein viral inoculant is an HBV-bearing inoculant.

10. The plasma or serum fraction of claim 7, wherein the viral inoculant is an HCV-bearing viral inoculant.

11. The plasma or serum fraction of claim 7, wherein the viral inoculant is a respiratory virus-bearing inoculant.

12. The plasma or serum fraction of claim 11, wherein the respiratory virus is selected from the group consisting of influenza viruses, respiratory syncytial viruses, and coronaviruses.

13. The plasma or serum fraction of claim 7, wherein the viral inoculant is a herpes virus-bearing inoculant.

14. The plasma or serum fraction of claim 1, wherein the mammal is a goat.

15. The plasma or serum fraction of claim 1, wherein the fraction is a serum fraction.

16. The plasma or serum fraction of claim 1, further comprising a pharmaceutically acceptable carrier, excipient or diluent.

17. A plasma or serum fraction for use in the treatment of a viral infection or related condition, which fraction is derived from a mammal exposed to an inoculant and which fraction has been depleted of one or more high molecular weight proteins, wherein the viral infection is selected from the group consisting of HBV infections, HCV infections, respiratory virus infections and herpes virus infections.

18. The plasma or serum fraction of claim 17, wherein the fraction has been depleted of immunoglobulins.

19. The plasma or serum fraction of claim 17, wherein the viral infection is an HBV infection.

20. The plasma or serum fraction of claim 17, wherein the viral infection is an HCV infection.

21. The plasma or serum fraction of claim 17, wherein the viral infection is a respiratory virus infection.

22. The plasma or serum fraction of claim 21, wherein the respiratory virus infection is an influenza A infection, an influenza B infection, and RSV infection or a SARS infection.

23. The plasma of serum fraction of claim 17, wherein the viral infection is a herpes virus infection.

24. The plasma or serum fraction of claim 17, wherein the inoculant is a viral inoculant.

25. The plasma or serum fraction of claim 24, wherein the viral inoculant is an HIV-bearing inoculant.

26. The plasma or serum fraction of claim 24, wherein the viral inoculant is an HBV-bearing inoculant.

27. The plasma or serum fraction of claim 24, wherein the viral inoculant is an HCV-bearing inoculant.

28. The plasma or serum fraction of claim 24, wherein the viral inoculant is a respiratory virus-bearing inoculant.

29. The plasma or serum fraction of claim 17, wherein the mammal is a goat.

30. The plasma or serum fraction of claim 17, wherein the fraction has been depleted of from about 1 to about 100% of the one or more high molecular weight proteins.

31. The plasma or serum fraction of claim 30, wherein the fraction has been depleted of from about 20% to about 100% of the one or more high molecular weight proteins.

32. The plasma or serum fraction of claim 30, wherein the fraction has been depleted of from about 50% to about 100% of the one or more high molecular weight proteins.

33. The plasma or serum fraction of claim 17, wherein the fraction is a serum fraction.

34. The plasma or serum fraction of claim 17, further comprising a pharmaceutically acceptable carrier, excipient or diluent.

35. A plasma or serum fraction for use in the treatment of HIV infection and related conditions, which fraction is derived from a mammal exposed to an inoculant and which fraction has been depleted of two or more high molecular weight proteins.

36. The plasma or serum fraction of claim 35, wherein the inoculant is a viral inoculant.

37. The plasma or serum fraction of claim 36, wherein the viral inoculant is an HIV-bearing inoculant.

38. The plasma or serum fraction of claim 37, wherein the HIV-bearing inoculant is the blood, plasma or serum of a subject infected with HIV.

39. The plasma or serum fraction of claim 35, wherein the fraction has been depleted of immunoglobulin and albumin.

40. The plasma or serum fraction of claim 39, wherein the fraction has been depleted of from about 1% to about 100% of the immunoglobulin and albumin.

41. The plasma or serum fraction of claim 40, wherein the plasma or serum fraction has been depleted of from about 20% to about 100% of the immunoglobulin and albumin.

42. The plasma or serum fraction of claim 40, wherein the plasma or serum fraction has been depleted of from about 50% to about 100% of the immunoglobulin and albumin.

43. The plasma or serum fraction of claim 40, wherein the plasma or serum fraction has been depleted of from about 75% to about 100% of the immunoglobulin and albumin.

44. The plasma or serum fraction of claim 35, further comprising a pharmaceutically acceptable carrier, excipient or diluent.

45. The plasma or serum fraction of claim 35, wherein the fraction is a serum fraction.

46. The plasma or serum fraction of claim 35, wherein the mammal is a goat.

47. A plasma or serum fraction for use in the treatment of HIV infections and related conditions, which fraction is derived from a mammal exposed to an inoculant and which fraction has been depleted of one more high molecular weight proteins, wherein the inoculant is selected from the group consisting of non-viral inoculants, Herpesviridae-bearing inoculants, Flaviviridae-bearing inoculant, Orthomyxoviridae-bearing inoculants, Paramyxoviridae-bearing inoculants, Togaviridae-bearing inoculants and Picornaviridae-bearing inoculants.

48. The plasma or serum fraction of claim 47, wherein the fraction has been depleted of immunoglobulins.

49. The plasma or serum fraction of claim 48, wherein the fraction has been depleted of from about 20 to about 100% of the immunoglobulins.

50. The plasma or serum fraction of claim 49, wherein the fraction has been depleted of from about 50 to about 100% of the immunoglobulins.

51. The plasma or serum fraction of claim 47, wherein the mammal is a goat.

52. The plasma or serum fraction of claim 47, further comprising a pharmaceutically acceptable carrier, excipient or diluent.

53. A plasma or serum fraction derived from a mammal exposed to an inoculant, which fraction has been depleted of proteins with a molecular weight greater than about 10 kD.

54. The plasma or serum fraction of claim 53, wherein the fraction has been depleted of proteins with a molecular weight greater than about 30 kD.

55. The plasma or serum fraction of claim 53, wherein the fraction has been depleted of proteins with a molecular weight greater than about 50 kD.

56. The plasma or serum fraction of claim 53, wherein the fraction has been depleted of approximately about 1-100% of the proteins.

57. The plasma or serum fraction of claim 56, wherein the fraction has been depleted of from about 20% to about 100% of the proteins.

58. The plasma or serum fraction of claim 56, wherein the fraction has been depleted of from about 50% to about 100% of the proteins.

59. The plasma or serum fraction of claim 56, wherein the fraction has been depleted of from about 75% to about 100% of the proteins.

60. The plasma or serum fraction of claim 53, wherein the inoculant is a viral inoculant.

61. The plasma or serum fraction of claim 60, wherein the viral inoculant is an HIV-bearing viral inoculant.

62. The plasma or serum fraction of claim 60, wherein the viral inoculant is selected from the group consisting of HBV-bearing inoculants, HCV-bearing inoculants, respiratory virus-bearing inoculants and herpes virus-bearing inoculants.

63. The plasma or serum fraction of claim 53, wherein the mammal is a goat.

64. The plasma or serum fraction of claim 53, wherein the fraction is a serum fraction.

65. The plasma or serum fraction of claim 53, further comprising a pharmaceutically acceptable carrier, excipient or diluent.

66. A method for treating or preventing a viral infection or related condition in a subject comprising administering a therapeutic amount of a plasma or serum fraction derived from a mammal exposed to an inoculant, which fraction has depleted of two or more high molecular weight proteins.

67. The method of claim 66, wherein the viral infection is an HIV infection.

68. The method of claim 66, wherein the viral infection is an HBV infection.

69. The method of claim 66, wherein the viral infection is an HCV infection.

70. The method of claim 66, wherein the viral infection is a respiratory virus infection.

71. The method of claim 70, wherein the respiratory virus is selected from the group consisting of influenza viruses, respiratory syncytial viruses and coronaviruses.

72. The method of claim 66, wherein the viral infection is a herpes virus infection.

73. The method of claim 66, wherein the inoculant is a viral inoculant.

74. The method of claim 73, wherein the viral inoculant is an HIV-bearing inoculant.

75. The method of claim 66, wherein the fraction has been depleted of immunoglobulin and albumin.

76. The method of claim 66, wherein the mammal is a goat.

77. The method of claim 66, wherein the subject is a human.

78. The method of claim 66, wherein the fraction is administered subcutaneously.

79. The method of claim 66, wherein the fraction is administered in combination or alternation with an anti-viral agent.

80. The method of claim 79, wherein the anti-viral agent is an anti-HIV agent.

81. The method of claim 79, wherein the anti-viral agent is an anti-HBV agent.

82. The method of claim 79, wherein the anti-viral agent is an anti-respiratory virus agent.

83. The method of claim 79, wherein the anti-viral agent is an anti-herpes agent.

84. A method for treating or preventing an HIV infection or related conditions in a subject by administering a therapeutic amount of a plasma or serum fraction derived from a mammal exposed to an HIV-bearing inoculant, which fraction has depleted of immunoglobulin and albumin.

85. The method of claim 84, wherein the subject is a human.

86. The method of claim 84, wherein the fraction is administered subcutaneously.

87. The method of claim 84, wherein the plasma or serum fraction is administered in combination or alternation with an anti-HIV agent.

88. The method of claim 87, wherein the anti-HIV agent is selected from the group consisting of reverse transcriptase inhibitors, protease inhibitors and fusion inhibitors.

89. The method of claim 84, wherein the mammal is a goat.

90. A method for treating or preventing a viral infection or related conditions in a subject, comprising administering a therapeutic amount of a plasma or serum fraction derived from a mammal exposed to an inoculant, which fraction has depleted of one or more high molecular weight proteins, wherein the viral infection is selected from the group consisting of HBV infections, HCV infections, respiratory viral infections or herpes viral infections.

91. The method of claim 90, wherein the subject is a human.

92. The method of claim 90, wherein the mammal is a goat.

93. The method of claim 90, wherein the inoculant is a viral inoculant.

94. The method of claim 90, wherein the fraction is depleted of immunoglobulins.

95. The method of claim 90, wherein the fraction is administered subcutaneously.

96. The method of claim 90, wherein the fraction is administered in combination or alternation with an anti-viral agent.

97. The method of claim 96, wherein the anti-viral agent is an anti-HBV agent, an anti-HCV agent, an anti-respiratory virus agent or an anti-herpes virus agent.

98. A method of preparing a composition useful in the treatment of a viral infection or a related condition, comprising (a) exposing a mammal not susceptible to infection to an inoculant; (b) allowing time for the mammal to respond to the inoculant and to produce one or more beneficial biologic agents in the blood; and (c) obtaining the plasma or serum; (d) processing the plasma or serum to isolate the anti-viral activity from two or more high molecular weight proteins present in the unprocessed plasma or serum.

99. The method of claim 98, wherein the mammal is a goat.

100. The method of claim 98, wherein the inoculant is a viral inoculant.

101. The method of claim 100, wherein the viral inoculant is selected from the group consisting of HIV-bearing inoculants, HBV-bearing inoculants, HCV-bearing inoculants, respiratory virus-bearing inoculants and herpes virus-bearing inoculants.

102. The method of claim 98, wherein the anti-viral activity is isolated from immunoglobulins and albumin.

103. The method of claim 98, wherein the anti-viral activity is anti-HIV activity.

104. The method of claim 98, wherein the anti-viral activity is anti-HBV activity, anti-HCV activity, anti-respiratory virus activity or anti-herpes virus activity.

105. The method of claim 98, wherein the plasma or serum is processed by fractionation.

106. The method of claim 105, wherein the fractionation comprises a single fractionation step.

107. The method of claim 105, wherein the fractionation comprises multiple fractionation steps.

108. The method of claim 105, wherein the fractionation comprises fractional precipitation.

109. The method of claim 105, wherein the fractionation comprises chromatographic fractionation.

110. A method of preparing a composition useful in the treatment of a viral infection or a related condition, comprising (a) exposing a mammal not susceptible to infection to an inoculant; (b) allowing time for the mammal to respond to the inoculant and to produce one or more beneficial biologic agents in the blood; and (c) obtaining the plasma or serum; (d) processing the plasma or serum to isolate the anti-viral activity from one or more high molecular weight proteins present in the unprocessed plasma or serum, wherein the anti-viral activity is anti-HBV activity, anti-HCV activity, anti-respiratory virus activity or anti-herpes virus activity.

111. The method of claim 110, wherein the plasma or serum is processed to isolate the anti-viral activity from immunoglobulins present in the unprocessed plasma or serum.

112. The method of claim 110, wherein the mammal is a goat.

113. The method of claim 110, wherein the plasma or serum is processed by fractionation.

114. The method of claim 113, wherein the fractionation comprises a single fractionation step.

115. The method of claim 113, wherein the fractionation comprises multiple fractionation steps.

116. The method of claim 113, wherein the fractionation comprises fractional precipitation.

117. The method of claim 113, wherein the fractionation comprises chromatographic fractionation.
